SEQUENCE LISTING (3 sequences)

SEQ ID NO: 1 - DNA, 1027 bp, Adenovirus
ccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagcagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggtta

SEQ ID NO: 2 - DNA, 26916 bp, Cairina moschata
gctatcactccatttcaaggaagggcaaaaggccggttcaataccaacatctgtgtagctaagtaggatgaaatagattaggtgaccaaaatatctgcttattcagcaggtgttgatccacaggaggttctacgataaagctccagtaggagttgtaccagcgtaattcctggagggcagcaataagtccaccatctacactccacaaagtctgatctttaggtacaaagagctgtgctgatctagaatgatgtggttagactgaatctggctctaaatttctgttattagagtattatgtattattaagtaagaggccttgcttcttactgctgcctaaatgaagattaactttacagtgaagcagagtgagagatgaaggggtgataatgtgtttgaaatatcagacttaatctggcaggtgataatcagttcattccaaaaacgttaatgtgctgaatcagattgacaagaaaatgaatcctaattagttgaaattaatctgaagagaactgaaagtacatcatagcaaatagtaactcttcttccacatcacaaggaacagattccactgtgttagtgatggaggtttatacaaaaaaaaaaaaaaaagaaaaaccagaacagctcttaatttatatacttattgaagcagattctatatatgcatatgtatgcacattcattaatcagcagtacatctcaacccttaaaagatcctggagaactgttgcttgtcaaaaagcactcaggttaggctaacatttgcagcttttgctctagcatacatttacattaggtgtaaagacaaggaatgtgtaaaagagggtaaatgcattcctatctacaatgttatgtatattttgttcctgttatattggctttacttccaaagttgtagtttgcagtattagtttctttttattggtatccttgcatatatatatattcaataaatgagttatgaatttcatatttgcatatctgtcctttttctgaaagtagaactttaaaactgacacttcagtgcataaaagcagtgttccctattgatgttaaagtacctcgaaagttttaccagaagacaagtgagtgcatacttaaagtatgctacttacttggtggtaactgcgtatttgtgcattgttcctgactttgtgtaatgggtaagttgctttgtatttaccacttgctaataatgtgaagtggctggctcactaaaggttgccattctcacctttcaaggacttcttgaaggctgtgtcataataatttagagtaagacagatactgagagcgcagagtgtaatatagttatgttgcagaagagcggatagacacaggatgcatgttagaaccatataaatgtggtgtaaaagatcagcaaaacgaagaattgcttggaagatgcttggttttgtccaaataaatagctgctgagagtgagagagtgagtgagtaatattcagtatttttaaagtgtcgagaaatgtaatggggaaaaatttaaaataaatgttttttgtttgttttaaaaggagctgtgtgcttttatgttgacatgttgatttaattactgcggtaggtatttactgtgccctgggaaacggtatccccagtcaacacagacttatttgtcaggaaaaaaattgtcatagatcatcttcaaataagagttgacaaataatcaaatcttgatggagtataattattaatactgtgattacatatccatctttgcaagggtttcctgaaaaggtcagttttaagtcttctattgtcaaatctgatgtttggagtagttcgctacatggtgtttgatgccatgtaaatagttaccagatagacgttttattttatgtgctgtatgtttttgtttttcattcagtcagctggaatcattgaaacagagaaggtttctcaagaaatatcctgaacctgttttgtggatgtcttcattgcaatagctggtcactgaaatcttggaataacgacagaggaatccataggcacaggaccaaacacctttcattgtcctcatatagcgtgatgttagggcagagtggtgagtacttcagtggctcctgtgtccatgctttaatgaactctacttaatctaccatatgtaagagacttgcagcacaggcaaaacaagggaagttatctttgtgcctagataatgtaccacatatggtaaacaattttcaagcctcagagacaaaagaaggatgctgtaaagtctcaagtctcagcttgtgtctctgtttccacctttctgttctttctgttcagacctctgggccagtaatttaaactggaaaattaatggaacagagaaactgtttgtgagcctatagaaagatcaaattgtgtcagagtaatgttgctccttctacacccagataaactttttgagcagaggagaagcagtgaacagagctttactttcatgactttgcataagaaaacatgggaatgtggtccgagaccaatttaaaaatagaggttttgaaaacttgtttggaaaacaaaacttgcttggaaaaccctattcagagctgtgaatcattcacagacaacctcttagggttgtagccactcatcagctgaatatgattcaacgatatgctgaaaaaaaaaaacacaaacattgttgtggaatgtatgaacaggaatgtagtctgtaagatgcgtgtcttggtccttcttctttaggtaatagtagaacacctttctgggaaaatggtatccagctgttgtaggcactgcactccatggaaaatgtgaagaaagtggaaataatccagagaaaagcaatgagagggatgacaggataaaaaatgagccttgctttaaggtgatgagagaaaagtgtttttcaaatctgtaagagagccacagagaagaaagaaagaaacagttctcatcattgcaggtaggacaagaaaaataggtttaagttgctgcaggaagcctttagcttagacatcagaatgggtaattgtccttgacagtaggaagaattaagctgtagcatgcacttcctggagaggtcgtggaatctcaggaattaggttttaaagggaatatttgtaggcatcttttggggctggtgcaattgtaggttggaggtgctggggataaggctaatggaaatccctaccagccactctaaggccttctgcaaggagtcagaaccacttttcggaagtaaaactctgtactggcagaggtcctgtgcttgaccttaaggctcagagccatgcatttccgttttcatcttttcacttaaaatagcacttgtggtagtaataacatgctcttgcggagtggcacaggtctgcatggttatgaactttctgtcaccacccagcaaatggcattttccactcttctgtttcagattttcgggagtacttccttactcccaaattcctgattgaatacgactgaaaacttcaacacgtttttagtgcacgaagtgtactttatacaaatgtgtgggactattgcacacaacttactgattttcttcactgtgtgcacatgtgctctgtgatgatacagagtttggggtgactgaactgttacccagtctttaccgaattagggcagcgatcttaaaccttcatctgaacttttgctagagatgatcttctattttgtttagacagggttctctgcttgcttgcatttgttctaaaacgacagtctggatgagaagaaaccaaccagggctggcactgccctaccttttattccctggatactttacttggcacatcacttggcacacatgcaatacccttttttcaccccggtacccaaagaaaagacgtgctcctgcacagactcagccaaattcctgcctgccagtggagggcgttacagaggcttggggcagagggaggaaactcactcccaaagcatatactaaaaataggccaatgcattgcaaacagctttgctgtgagagcttccttctggcatgtagccatgcagctgccttatagttttgagcctagatacctccaaaacaaaacaaaaagtcgctaaagttaaatgcagccagcagtgcttatagtcttgataatccctacagtgtatcaattttagtttcttccagagtgcaggtagtttactcaatttaacttcaaatgtctgagttacaccattgacaccattgtcaagactagtgctggcaatccctgaagtctgccaagttcaaagcagggaggaggcctttaaagttgaaatactcgggtgacgtgtccaaaaagttttcctgtggctttcctgtggagtaggaactgcagtagctaggttgcaaaaagtttttaaactctccaaattgctgctaactaaacatccttaaatcctgacatgagagtatgaaatgagaaagtcagttccttgatttagactagcttgaaaagaataaactttttgcacaggactccctggacagttgggctggataatagaagtgattcattcaggtcttgatgcctacaagtttcttgggttttgccatattttccagtgatttgtgacatcatttttgccctaaaataaatcgcatcatgtcctgccgtatgttttgactatgaaaatggaggtttcggtttgaataccaaacttcgttgttacttctttcagaggctgttgataaaggaaaacgtgaagcttttatgtaaatgatcatgatttctcatttgcatcaagaatattttccaaaatagtagttaaatccctccaatgattttcagctatgtcacataaatcaccgaaaacacggtaattttgcacgggtgttttttctccctttttttattgtctaatactcagcttatatttgagtcttctgtatccaattacctgatttgtgtggcaattaaacagaagatcagcgtcattgtacttcacattctttttaaactcatcagtagtatgaccacactagttggtcttgtaagtttgtaatttaagatgtgcctggaaggggttcaaagagacaggagcagtagtgtgtctcacttccctttggaaagcctttctgatactgtcaagcaatatttacatcaaaagggaactgaattctggtaaacttttccagactccaagtaaaccgaacaacctgagatttagataaaccttggtggatttaaaaacttgatggtatcactgtctaagacttgtgttagcccatcagatattgcagacgtatattttgaggaaaggctttcagttaattgataaaattaagagcagagttttggcaaaaaaaaccgccatctctgctcagtacttcatggcatcttgaatttctgcagttagaggtacaggtgctctgttctgcaattaacccgtgctgttaattaaaatgtactgtttgctaaatatgattagtgtctggagccaggtagcttgaaaaagcttcaagattatttctttttaccaggaccatcaagtttttagccctccctttagaggaaggttcagaggaggtcctatttaatctgtcccagaagaaaggctatttctccccttagaatggggcttggcagtccttttcagagcacttttcatccttgtctcatagggatttttaaggacagatttctgaaaggattttccaccgggtaaagcgggatcctgaatgggtgacagataatacgtaggaattctggcttcctactgccaggttgtttatcttgagatcttaaaaatggacttatttccctttttaagtgtaaactgagttcttgaggcaccttttccactgaagtatctgactttgcccttactgcccatctcagtcttgcacatttaacccaaattcagatccagggtgtggtgttactcctagaacagctcttactgtttgaggatctgaatgactattttgccagcttgtgccatacaactcgagaggacctgcaaaggggaggggagtcctgatgggcagttccctcccagcctggcatgcgcttcgtgtctccgcttcccttataatctgcccagaaaagatttctgaaatacttgagcagctgccacaatctgcatgggaggcagcagccttggcatgtgagctctgccacctgatgcccggtctgtactgaggcagtgccagctggatctgcaagcctcagcgaatacagccttcgtcagcgagttctctgatcagattgctgccttggagccttttgctggtcttcctcagctcttctcctgttactcagatagccttaccacctcctctcatcatcttttgagtttctatcctcacgttacagccaggtaagcccatttacccatgctgatatcaacaggttccagattttttaaaaaacaatcctgtctggctcacagctagggagcctctagtgacacagctaagtcccctgcagccatttagaaatgtttctggagggatgcaggtacgtcttgcactcaggtgcctaatgaaagcagattgacctgttcattccttcgaaagcaactaagcaaagtggggaatcccatgtctctgccccacttctgtgttcctgacggcaagacctcctgccagcaccaaccgcctgtgtttggagtcagccagccttccgttctgactgaagttgaaggtacagtacaaactgctggctaaaagaagctgcttgacttgcttttttaggaggcttgtcttgtggggaaaaaaaaaaaaagtacagataaattctgctgattccttctgagtcacccagcagcgatggcaaagctaggagaaaaaatgggatcacagagggattgatgaatggacagtgagtgacctgaagatctaactcaaaggacgttacttcaagtgcaagtgtcaacatataaatgctgagttgttgtctccaagtaccccactgttggggtatcctggttaccaagcaaggttttagtaagtctgttcgaaaagttacatacagctgacattcatgcaggccatgatttgccaggcctgagctctgaggtattcctggacggttagagaggataaaataatttagaagtcaggaaaccactcagagacaaaccattatttataaaattattttgccttctaaaagatgtgcgttccagagaaatcacaacgttggctccaacctttttgcatcatctagctagaaaatgtgcagcttccagataccgccagatcacctcccttgaccctgccctggaaacatcaatggctcccataccagtaagaacaaaactggtcatctactaaatccgttgctgatctcagctaaccaacttcctgtaatatcagcaaatatttctgcttttgtaaatctgttcttccttccataatggggtgccagcaggaatgcttgggatccagaaatgcgctggttggcaggaagaacaaagaaattgttcacccagaagggcaaaaaaggactaatgctttcgtccgcttgggagaagttgcacagaacttatgccaacaacttttgcacgcaatcagaaagatgccgctggatgttacttttaatagcagacgttaatatcagttattaattagaaatgtcttcagtaaccagactaaaagcagatcctgaaacactcctgtggttgaacagtctcttgacagacactgcccactgtccagtaatgtcaggcgctctctgaacttgacagggcagctgctgtttttcccagccttctggaaataggccagctctgacatgtttctgatattagctgggtgtatttcattctgctgccctaggcagtttgataaaggctccttcctcctgctccagagctaacccacccaaactgtagccgagcacctcgctgcaacaaaactgcactcgctagggttctgcctgtttgcttcattaagatctgcttaaattgtttcgtacaaaggaacactcaaactgatttctgagcccaaagtagcagtgctaggtgtacatcaggagttgtttggcatgaagaaacattgccatggcactgtatgaataaagttatttttaagaatcattatcccttccttgataccaagtctttatgcggcagaaaatcaaacttggtctccacccttacagaaagcagaggaatgctttcagctgatagttgcttaagctagaatataagaaaccatgaatttctgtgtgcactgcggcattgcccttcattccagacctacagaaaaaaacgacacttttgttactatttttttccttcccatatgagaccaggggagctacccaggcatttccattcttataattttacctcaagatcaaattttctccaggcagttaaaggcagctgcacccggagacctcgctcagcctccccttgcatcccacggagctgcgtttagtgagaaacctcccccgaggtgacgggctgcaggggaccccttcccacacgcgtccccgtcccttttctcagtgcaaacgcagccaccgccccttgaaccctcctccgggctctctcggttcggcggaggcaggagggggccgtgcccgccgcccgggagctcctcacagggcccgggccccggggcggagcggccgcggccatgttgagggcggggagcgcggaaggcggcgccggggccgctgcggggcgcggcgcctccccgctccccgccgctcctcgccgcccgcagccgcaacaccggccccggccccggcgggccgcgccatggcgacccccagcccctgcatcgtggtgagtgcgggcccggcgctgcctcctcggcctcccgctgggcccggcgtgcggtgaacggggaggggggaggcggctgcggggagccggcgggggccgcccgtccccgtggggcgccccgcgtggcgtggagtcaccttgaggggctcgggctcccgctgccctgcacggccccgcccggcccccagcgcccctcctcggctgccggctcggtctcgcctcctgcccggtgtcctgccatggggccggcggggtgttggcaatttcggggcgctgcttgggcggctcgcggttgggagagccgcgggtgctgcctcagaaggcgcccagaaataacttccaccagaaataacttccaccaggcaccgacccggcgtggcacaacttcctattcctgttgctcctcacagcgcgctcgggaggtcgtgctccgtgtaccaaaacctgcgagctcagcacccgaactgggatgccttttggaggaagaccccgctgagggtgtatttttcatcatcagtgtgctctgctaagaatccttttttttttaattattattttttttaaacccagccccgggtgccgtgagcggtagcgtgacacgcatgtggcgcctcgcggcccacggccatgcctgccgtcaggctgtgcccgcgcgcctgcctgtactttctcgctaaaaatccatctgtaaaacccaccgaacagcccgctcagtaccaaacgtagcctgcaaggcttagtggatgtgctagatgtagttatggggctgaaccgtaggaaatttccgtagctgctgagagggacccagctcctgacacgcagcaggtcacgggctcggtgcctggcctgctgcgagcaggggttgtgcaggggctgctgtggctgggagggcaggcctggctcccggccaaaatcctcattaccgcttgcgcaaccaaaaaacatgtaaacagctggctgatcacaggcaaggtaagtgcttccttgggcactgaaatggaagcgtgatctgcaccggcagcggagctgaggtggatgcttaccatgcctgccccgctgcattgccagcagcaggcttcagtgttccccacaaacttctttttacactgagcagccttcagctgcgtaaccaacaagtgaagcgattcaaaatatattgtcattttcaagtggcagcggggctaaggttcaggggaagttttttcttggtgcttttggaacaatatgccctgaaccacagcagtggttgtataattacgtacataggcaaaccccttgtcctaattaatacactcctgctaatgttgcaagctgtttcatgtggaaaatctgcccgtgcggaacaggttgctgaaacagggctttaatttctgattctaaagatcttgaacctccactgtttccagatggtggtcagagaaggcttgtttcataggcacataacttgtcccgttaatatagctgtcttgtcttgtattgacagctggcttgtatgttttgttgtgattcggtgttgtggagatttggaagatgtccctgttacaaggtctgcttacttagttgcaaaggcaggttttgcattacactgctgaaatcagagatgttgaaaagctgaaagctgcttgatccttgtttatttatttatttttaaatgaaataagattatttgaggtgtagtaatttcattttccccgctctccaatggggggaaatgttgttagggcttgaaatcatttcccctccacatcgtgctgagcgacagctcatgtagcatttcagacaactcttgtgaactgtgggttgtgcaagcttccactttgtgctctgttcagaaggaaaataaggtgatcccaactagtgtaatggatttggtagtttattgagtaaagcaaaggattggcagtttctcactacaggctttctataagactttgtagaaatctcaccttatttccttttcagattgacgatgatgaacaaggttacgacctggacttgttctgcatacctaaacattatgcagatgatttggaaaaagtctatattcctcatgggctcatcatggacaggtttgtttgacttcagacagtacactgctccagctgattccatgacactggaaaaaacaatcttccagtgatagttttgctgcctagtgactgtctaaacagattacatttaattagagactaagaaatacacatgttaattaactctctcttgtttggcttcaaagagtttgtacatttgcagttacgctattgtttggaaatttgtcaattctcaaagaaatttgtggtacgtagcagtctgtgactttctttacagtgtttctttgatgttttacttaaagtaattagaacacattacttgttgtgctagttcaatctaaaaacagttatagtctctcaaacatctttaggatattaataagagtagattattaatcacattatgataagactttttcatgttcatgtggtagataactaacacccaattttcccctactgtctgcgaaagacattagcctttgcaaataccaatctgtcacttgtggttgctgaaatgtatgattttctctggaagttttatctcgtgatgagaaatgggtacatgaacctataaggtgtttttgttttactttgtgtaagtaaagtggaggagttgctggagacacagaaccactgaagagcggttctgagtagatcttgtgaataggaatgcttctagattttgcatggtgctgtttgatcagattattacagtatttataataaaatgttttttaactttacactgaaagaccctatataggaaagcattggacaaagtacaggtcattaagtagctgatgtaaagtttgtaatggcaggcattctctgagaaacctgctgtcagctgctatactgtaaatacataccatgctttctgaattaaattgcaagataaatttagaaacaatgatcactgaaaaactgttcagtgttctcttgctctgctttattggcattatattttgcagtcaggacaaatttattcagcaaaacataacgctatagttgataatttgagagttttcttgctcctcagttagtgagagctgttgtctttttggttgtgctgatttattttgctctctgcatggaagctgaacctatctttggaagaagaaaacacccttatgtctcttatctgacagtaaaacaattcagggtgttcagatttgctttggctgagtatgatgtatgaaaacaaagaagtttggcagtgttactgttagattaaccttggaacgcaaaactttgttgaccaatagtgggttaaagtgactgaagcattaggcaaatatttctgagcaaaatatgcttccgagtttgcatgtgtttgctgttgttgtttgcaatacaaaatactgctgccatagtaagcaaactaaatgtgttacaacagctaactctcttttttttttttttgtataggacagagagactggcacgagaaattatgaagggcatgggaggacatcacattgtagctctctgtgtactcaagggtggctataaattttttgctgatttattagactacatcaaagcactgaacagaaacagtgacaaatcaatccccatgactgtagacttcattaggttgaagagttactgtgtaagtatctctgcaataccatgcaatttttctgtaaatttgactaacttcaaattaacaacagggatgattgagaattgccaacaaatgttgcaaaagctttgcctaagtactgcctaaattgtgctaattttatacaaatagttaagacaattaaggggaaaaaagcagtcacaagctaacttgttctttgtctatcttatatgatctggtttctttcagactttatctcctcggcccagtaaaatccagagcaaagagaccctttccatgtcctttacttcttaaacaatttcctctctgccccctgtcccacctctaaaactgtggttctagaataatacagaggatagtcctacaaatcttattacaaaaacttaactctaggaattttcatgtggcttagacatcactcaccagataggaaaacttgaaaactgtgagcatggttatatttgggttccctacctcatcttttgtttggctcaactgaacaatgaatggaatgaattgcatttggccatgaggaaatactagattcataaaaacagatttatggttagcggccactgaatatggtgctaccttttaaagcctaggtctaggttgccttggctttatgcttttggaaaagaatttattttcattttgcacacaagtatttaactttacagggaaatggagcatgaggtagtatgtaagatttttataagggaagcattagattacattgtgcaggtcaaaggaggaagcagatgtatgttagtactgtatgcttcctgactcagcaacagcctagtctagtgttttcaagacagtatttgtgggtaatttagattttttccataagatcacctgtagtgtggaaataattaagttttttgatttcaatttacggtgtcaaaataaattgcttcttaaggtcatgctgttagttctgtgtctcgatgactgtgttttccagtcagtagtataagctacttgtcatggttgtggagtagtgcctgctgcagtagaaaaaaatagattactaagttggaaactcaactgcattgttcatttaattaaaccagagataggggtacttgcagatttaggttggactgtagaagagcttcaaaaatgcgctgctgctgttgagcgatctgtaaaaatttcacctcgttgagtggcaaagctcagaccagtcagagtttggccaggcctgtttgcagaaaagctgtgccagctcccaaggtgtgtgtgcagtgatgtcctggggctacctggaaaccaccgtcatgtctgcagagcgtgctgcagtgactcagctgcactctttaaagagaaggcaaggccaggcagaaagggacatgccagcctttcgttacatcttgcaaggggagagctgcttgacacactttgtttctgctccccacaaaagattcaggctccctctaccagtccattcaaccacaaagccactacgtgcccacatacgctgtctgtaaaagccacaacagaaaggtgaatgtctctattagaggataatatttgaaagaataggaaataactcccaccttttttggcgaggaaatttgtattttctgctttcttcaaggaacacatccaataactttggtttatttaaaatataaggactacacccttctgctctctccaccttccgagtaacttggaacaagctatgggataagtgttgtgaaacacctggaaaaaatactacattgcaaagcaaggcttattgtacagcatgtttttattgttcatctttatgcattaggcatgtattggttctgctgcttttattttttacaccgcccccccaaactatagtgcttttatattaataggaaactgaacagcaaaataattacggaagttttaattctctttttgtagtaagaaggagtaaacaaagagaaaagcagagaaatttacacagaaaggagtgtaagcagaatgttttccatgaggtgttggcttcagggttttggttttgtcagccaattctgtaaaggaatgttttcttgttctatatgaccgaggagctccagttacttgatggccccactagcagattattacgagcatttcatttctgtcaactctctaaggtagttattaatatccagacttacgttctttagattgcataacttgatagactgggtaagactgtcctaacacaaacatgcgtgtaattgattcacttagttttccattattaatggtgctggaatgttaaatctgctctgtagttttcattcgtgctttactaaacggaggagtcagcattcttaatgatgggaaaaccatcacaaaccgtaatggacagttgaaagtagagctagctctcttgtgattgttgttggatgcaattactagatcacgctgcctgcctttacagactgggtgaaagatgttaaaaataccgtcgttgcataaacaaagcagctgaactcgtataatggttctaaaattgatgcgattgatgttctattcctatttgaaattgatcatttaatattttcaagcttatgggttatttgtttcataatgcaaagttcatactatgattgcacccaaaaataattctctaagaatactgagatgatgaagctgtgattaaatgttgcttataattcctgctttcggagtccttacaaatgagttttaatgatctggcaagctgttaagaaattgaaagatagtgtttgtgaacagaaccagatctctgtattcttgacaaatagtttatttttcttgtgctagaagaatggcttttcaccagtttcctgcatgtaaagttagagttaagcaaaagttgcttatgtagtcacaaagggcagccaaaacacaatggacgtgttgcattgtcactcagggaaatagggcttttcggaggagtactgcagctttaaggcaaaatctagttaggctggttgagacagtattctgaaagatctcatgttaactgagccttatagcaaataccatgaggaacttaaaatgccttgtcaggggttacgatcttgcagcatgttattattttccagcaatgacaatgaggcagtgaaaacagatctaaacagtaaatctctaatgtgttaaatcacaaagcattaaagtcttaccagtactgaaatcagaaataatgctcctgaagataattaaagtgttgcttaattattaaacaaagttctttccaatggaaatctggttaaaaccagtgttggaaatttgttaaccatttttgatcctttaattgctcagaaactcagtacctgcataggcgtgtaggtttatatgccagcgaaggtttatgttttgtgttaaaaaatgtagcaatttgcagggtgcttggtgggaagaagaagggcagattttgttggttagattgtttttctctgctgatcagtgtacttgagtatgcataacccacagggttctgtagtgatctgtagttctgtgttgttattgtttgattatagtgtgttgctttgtagccagagagatcaccaggaaactgatctgcatgcatgtactgagaggactggtctatcatctgtgcatggtgttttaatggaacaaatatctgtaacatctttctccatcttccttctgtaagaaatggtgctttttacatccttccatggaaaaaggacgaaattcttattagtgtaggaatagataagagagaatcctgcaacataaacagggaaaagcttaaagctgtcatttaagataattttaagatatgatgaacgcaggaaatgcccctcagcattacaaacacggtgcttaactgaatcccattttcgtgattaatctaaccagtatctctggcattcagtagcagtctgctgtttactgggtcaagcagcagacattttggacatcctatttttggactttttcaacacagtggtattggttctgttcactatagttttctttaaaacatggggtaaaactgtaaaggcgctttttctaatgtgttgtgcttgccttttttttaatgttttgtcatgggaatctaaaatagaaagtatctgttagtctcgttatctgcttttacatacctagagctgtcagcacttctttatcctagagcattgacgtcactgtccaagggattttaatacattttctctttactgtaatcccagtgtcacttatggtgtgctcagtgtgtgtttctctctaaaccatttaccccttttttgcatgtgataacacttctactttttgtctgcaactggtacagtatgaatatgtgtgtcagtgaaaacaggaagctgtatgtggccgtctgttcccctgtggtctcctctctattgtatctgccattttggggcgtggggaaggaactgctcttttgctgcttgtctgcatggtcatgcagtaaggttgggcttcagactgaagcggcaaaataataaaggttttctaaagggaagatatgaagtgaagagctgtgagaatgaagtttgaaaagtggtatgacccttcaaataaatgattaaattgtaggataatgacatcatctgtaagtagcacagtaatgagtatagcacaattaataagaactaggagacaatgtcaaggtttaaagtcagctgaatgactttaaataaactgcagagatctggaggtaccccaggagttgtttttaactttcttatataacaaacagttcactgatgcttacatcaaagcctagttttactaaagcaaaatgctgtacttcagtgataggattgtgtagctgtgtcgctgtttaattaccacaatgagtaatttatcactatttgtaaaataggcaaacccaagcttttctggctattcttgtagcttatatttcttgttcttaatctgtgccttgtttgctggctatttcattgttgttatgattgcttatttttattcagagtaaggctttctaaggcatcaaagtttaaaatgtaaattaccagctatatagcagcgtacagcttggaaactttttttttttcctgttttccatacctttaatgaatatgtaggaaataatatttttttaatatacatgtgtgtatatataatggtgacagatgagaagtggtgcctgtatttatgtaaatattttttcaaacaatcttttatctgaaataccatctttcagtttgattacttgtaatttcttagttaatagatgttgctggtgcaatgcttctgaaagctttggtatttgtgggttatgtactttgcagttttgatatttattagaagaaaaattactttaattctggtaatactaatgcttaccctgctctttcttgctagaatgatcagtcaactggagatataaaagtcattggtggggatgacctttcaaccttgactggaaaggtatggcatgtcatatgtttaaagcaaccccctcccccgcagaaaaaaaaatcctaaaaaatcctaaaaaaaaacaaaacaaaaccaaaaaaaacaaaacaaaacaaaaccaaaaaaaaaaaaaacaccaaagaaccaaataattagagttttagaaaggaaaaaaaaaaaaaaagaaagaaaactgtagtgtttcatttaaaatttaggttagaggttggacttgatgatcttgaggtctcttccaacctagaaattctgtgattctgtgattctgtaaaatattcaccattgtagagtggtggggtgcataagccccaccagagccatcagtttagtttgaacttcataacatatgtatgtatattcactgtcttcagtatcaggaagtgttagagttgtttttctgcattgcctaagaatagtcttgtcttggttgttgttaacaaagtacctttaaaagagagtagcgttacttattactgtgttggtgtttcattgcaatagtgaacagagttggtactgtctagacatataacttgcaggttagggaataagggtcctgttacaggcaatgaagagacagaaatgcagagaatgtaaattgggaatctgcttttcttcagtggctgtgtagagatcattgagctagatatctgtgggtaaaacatggatgcctcctgttctgtcagaacacatggctttccccttctgattacacataaacagtctctgtcccatttcagtgtaagtgatgtaggtgtgacaagacagtacataactttatagtatgacattctctgcattataagaatttaaaggccactttgtactcagcatgagatgttgaggcaagtctcattgtcaactaattgtcattagagcatcagaaatgttaactaaatagaattcactgttatgtgtcctaacatacataaaccatggtacaaatatttaactgcattgaaaaataaattattaagttataggagacaatatatgtaagaaatgctgtattctttaaatgctaatggattcttctttttttttttttctttccaccagaatgttttaattgtagaagtaagtattatgtttcactttgaagttctaacgtgtgactggcaaaagaccttaaacagaagttggttttaagtcagaaatcctacagtttctggtttcaaggtttttagtttctgactctaggcctgaattatgcaagtggtctctgaatttgaaattacctgtgttttgaattctcacatagcattcaactataccaaatttaacaaggaaaatatctgtgtaaaattgcttgctgtttgcatttgatttcagttttctgtgattctgtcaattcttaaatgtcttttttttttttttaataattcttattaagaagtagcctgagcaaagtggtactaagtgtgttattgttctgagtttataacagcaaactaggaaattaaactggaggggtagaaagaagttcctagtaggaatatcctaaaatacatgggaaaacagagctaaggccaacatctctgtgtacagtttctttacaggcatggagaatgttgattcctaatttgcattttgataatgcttaattcccatttctctatcttgtcgtggttctttgatactaggtacaatacacttcagtctgatttcctagtagctactgttgcttaaatcttgcttccatacctctttgatgaacgatgttgcttacattcagtaagtatgacttttacaaaggatgatctagaataagatcgctaacttgaggttggcaaagcagaagtactaaacaggaaaaatgaatgtatagaagtaataatgtcttcagactccaaatagtggaaatggctttgtccataagctatggcaactgattcagacaccaaatgaaaggatgtttccttctgtcattaaatgtgttttttttatgtacgtctttttttaatttgagaaggttatattattccataggaaacacttttgacctaaactgttttagttcagttctaaaactcctaccaaactagtatgtatacagcagtcatctgtgtgaccggtggcgtcctttgaagtgtatttgcttcagataggcagatgcatttaattacgtatctgctccatttcagatgaatgtatgtcttcaaagtcccaggcagctaagtaggttagtttctcatctgttcagtgaatcatctctccagtgccttatatataattatacattgtcaaatatgaaccctttgaccagtcaccgttttaactgtcaaacaatgggggaaaacaaacaaacaaaaccaaaaaacattattataggcaccagagcacttaggttttccatacagaatccctttgatgtataaaaacaaacaaacgaaaaagaagaaaagaatcccatccctttctgtgcaggggatgggattaagatggtgtgtcaaacatcattttagtgtatataaatagaaactttagaactgcacccagatattgccagtgcagcttctttgtctttggatttttttgggtctgtaatgtaagttgcacttctttttcctttaattgcattgatgggttgttctgaatgttttcccgtcttattttctttctgttgtatacatctctctctgagttcaactgcaagactaatgttccatttataatactggaattaaacaatgtttgtcaaatagctacaaggaacacaagccataaactcttcagagggagcctagaaggtcatagttcagtaactgctttcaaagtagtagttatctacagttttgactgttgtgatcactttactgagcataattgcttaagcacaatatagcgttatgtatcacctgtatttgattatgaacattattttgtcatatttttgtgctattacggatgagcaatttctgcagtaaaaacttgaaaatgctactctgaaaattttaatattctcacttacatgacacttttattttcaggatataattgatactggtaaaacaatgaaaacattgctgtctctactcaagcagtacaatccaaagatggtgaaagtagccaggtaaacttctaaatgtgattatttgtgctgtatttcaacttcagaatgacacacagtggaataaaaatataatatagaatatattcccatagaagagaaacaggaaggaacatttctgcaggaatcatacatgatggagttagagttcagtatttgtttaaactcaacttctgcttgttgtgggagcatatgttcattaaggctaacagaaatgctcttctttccagtcctattgcttgaaaaatatcagtgccttatattctagtaatatttgtggggttttaccactgatctttaacttaggtttaaaagcatatggaagaatttaaatattttgggcataaaaaaacacaacaacctctcaaggtggcaggggtattttcaacacaaatttagttcataaatataaactttataaatataaagtttaactagagattgggattaaaaaaaaaagtcagtggttgacgtctatggacatacagtatctgaacatgtttcagtttgcaacttcattttttttcagtggtgtatctgcaatggagacctttggtctgccagctttacttgtagaattaaattagtttgcactaaacattaccgtttatatagagtatatagtgtagctgagcctcagaggtcattctgtgagtcacccagccatatgctcttttaggcttttctacttagttgcatctttaatcagttagttatgaaacgttttctaattaaataacttacagttagtaagaaatgttgagggtaatattaaccaatatcaacattcctcctacttctttttaaattgcatcttttatgctcctatgctgtgattgtgtattttacaataaaagcataatgattgctggggattagagataaaccagaactgtgtgtaaaatctaatcagttgtgttgccataacaatgtcaaggcagttgttagatgtactagagcagctcttcttgatgataatgtggggatgacctgctgaaggtgaggtttttgttttttttttctccgtggcctgcttaaatgtaccagattttaaatatttgtcactgaatagtaccttaacgctacagtgtacatcacaagtagaatgcagcatgaacaaagcctagcttccgattgtttaaacctgttgcagtttgggtctgccgaagacctgagcaagtaagcgtgatcgtgctggatagctttaagtgtcatgctcttagttcacttgcctttcttttcttaattttgttttctttctctctgacaattaagcagttgtagcagggagatgtgaattaatgctaatgtgattttgggttggtttgatcaagttttatgccactactcaaattaagactctcaactagaagtttcagaaactgaatgtctaaagacagacaaatgtcaggatgatggggtcatcttctgaaagtgtgcaactgaataatgagtgagtcaaattaaactagaaaggtatttcattctgctacagtacccccaactctgcatgtctatgctttttacacctctgactattaattctgtgaaagtggagctggtttatatcagcttatgatcatcaaaaccaattacattttctgtatgggggtaattagtactaccagtggaaaaacagctgaggacatgttgcacaagtgcaattgaggttacatttaaccagctgaacttttgcttcataacactaacgcttttgaaaagacctcagctggtctggctagatcccaagttgtcacctctttacctgcgagctaaactgattgcacctgaggtttctaaaaacacataaaacctaattaagtcacttctggcaatgaaaatccttccagtaaagtatctcacagcctctttgctgcaaacattttgtttatcccttaggctgtaaaccattcatgattctaggaagttttgagtttgtctatatgatcagctgcacctgcaaatgaataatatgtttaaaatgtatatagttatgctgtcaattatacacatgtagtctcactgaagtggaattcagctaatcttcgtgtttcatataagtgaacctggtgtttttccacgtttcagttttacattgtttttatttgacagtttgttggtaaaaagaactcctcgaagcgtgggatatcggccagactgtaagtgactttttgacacccattatcagtttttaaaaatatgttgtatatatgttgatgtattgtgtactctttggtcaaagtccaagggtgttccaagtaagttttcatgaatggtttttgatttattttgctcttatttgggaagtttaaatatttggaatgtattttgacagagttaatacagatttggtctagtcctttttttcttaactttttgtggcattttattatacaaaaaggaaactttgatattttagtccaagttaatccctcttcatatggaaattctcctagcttgcacgttaatcaaatggtttggtcagcagaagtgtcatctttgacacttttgggagtgttccaggtgcctaaacatagccacccagtgccactcggatgtccccagaaatgtaatgggaccacacagggtaacagatctgatcaaagtttacagtggtaagatgttcaaagtagcacaaaccccatcttgtttaagcatctggattactcctgtcatgtcttcttgtctgtcccaagaccataaaggaaggaagagcatccaaagcacacgggggtgtcagtgatagcccctcttggcttggaaagggctacgtgtgctatccctgtgccagaggttatttcctgttcatgtggactgtgcgctttcacaatggaagtttaacagaattgcatcttctttcccctcctttccaagtttgcacagtattcttaaattgaaatttctcagtgccttgaaagcttaattcaggtgtttccctcactcgaggatgcctctctttcctgagatcttattatgggtttagctgggttaacagtctgcctttctgagcatcttgcagcagatgacttgttttgccatctgtccagagacgacctgttcagtgcagaacccgtgagctctgaaggcaagagacaacgcagcagcagagcttgggcctgccttctgtccttgtatcatgaggccaagtagactctaaacattgccttattttttccagaagcgtacacagtctttcactggaattgtgtgatttatctccgtttttttcattactgagcctcgctgtaaccagggagagagaaacagcacagctcgggagtctttcatgtttagcataaggggagaaagccttctggaaacgtccttaggttgtcttacagtgtgtgtggatggcttggactgatacttcttgactatccaaggactgttctgctagagccatgtccacttggactatctgtggaacagctgatgtgttttttttaggcagcttctatgggaaaaaaaaaaaacgaaacaaaacacaacaaaaaacccgccggtttctgaaaagtgcaagtgacaccgtaaataagagagcgccttttaccctgtggtcttcactattctctataccaagggcttggcacttacagcgtgttgctctgcagagctgtcccgcagtcagagtatgtcctggtacaccaggttgttgtccacgagcagccagaggggagtgaattttgtttggcactgcctcaaagacaaaggactggttcccctgcggtaactctggttcctcctggtgttgacgtgttccatgcttactctcccagctcttcacaaagagcccagttgggcttgcacacctgtagttttgagggagctgagagctgaacctatctgctcgttatcccagaggtgaccttacttatagctgtgcccaaggcgagggcacactcacacaggtgccaccttttgaataatactgtgagggtgacaactaaaacaaaataaaatgctactgcttgggaaccagtaagtaaatctatttcattttgttttagttgaattaaatcaacaaatagagttagaccattaactagcttgaatattaatctcgggatacttgaatggaagaaatgaaatgttatgcttatatgtctataaaattctagattctctctgtaaaaaggtctagtgaactctaggaagttcttttctaacatactgatgacttatgttctttttcagttgttggatttgaagtgccagacaaatttgttgtgggatacgccctagattacaatgaatacttcagagatttgaatgtaagtaacttttcccgtatgtttttgtccatttaaaactagggaggaggaaggaagggaagtctcctcgcatcactctttctcttctgtgtaaatcacagctcatgtgcaagcctgttccattagtgatgcaacgacgagagcaaaggctagaccagcacaacagtgttttggagctcatatgaaactaacttttgttttgcttcatgcagaatggaggtattctgcatgaagctgtcagctttgaaatactgtggcagtctgtatgaagtttcttatcttaccagacatatctgcaagaaacaagtgcatttccttagttacatcaaaattagagaggagagaccttggatgttgagatgtttgcattttatgctaggtcattattaatttttcatctgggtctcactaaccttataacagacatagggtaggagagagagagaaagtccagtgttgagatctgtagacaaaaaaaaaaagaagctaaagtttttctgcataaacaatatcacaaggctcttcggtactgaaatgtgtatagcatgcttttatttttttcagaagtgcattattgaatacatctgtaaacatcatcctacattactttctaacaggagctttttattttgttttgttacagcatatctgtgtgatcagtgagacggggaagcagaaatacaaagcatgaaagcatagttcaagtgctctgatagcagagctttgaaatgttttgtttactgagtcctatctttcacaggcttcaactctggtacaccagctaaaattgtagaattatccaccccccttgtcatatgcttactataacttattgcatgtacaatatacaaatcttgtcttgttcatttatattttagaaatgtaaacaattagtgcaattctgcactccttattttgatttgcactatgactctaccgactattgctgcccctggttgggttgtgctgtttgtgagctccggagtctaactcttgcagtggtaaattgcttaaacctcatcaacccagaactgaaatagttcaagtactgtaaatgtaaaacactttatgatagggaagtctattagtaatatttttaaaatctgtaatttaagttttatattttcatgaacaagtgtgattgtgattaatggatagttgcacctttgggtgttataaaacatgaggagcagccagttacagtatctgtaatcaccaaggagctgattcaagcctcctggagttgccttatcgctgtaaaagtccgggtaaaaaaaatctttttttttttaattttttttattttttgagctaaaaggagatggaatgacaaagcagaatgtttaaaatctagtttaagttgagctattcatttcttttggattttttctttaaaaatacccatcagacagtgaaattgccttttaaatttaaacaattttaatatatttttgaagaagtattgtaatgtttactttataagatctaaaaaacaaaaagtactttaaataaaggctgtctctttaaaataagccccatacatctatgccactcattctgtatattaaagtgctcttgaaattttcttagtaatatttttcataagtgtttctgacagtgaaggcagactctacgtttgttatctttgtagagcctttagtcagtattgtgccatctacaagagatgataaaggcctgagggaaagctatcgaaatacttgcgtattctataaccaaactggatttcagagaaaatgcactaagtagtgtcccgtattctgttttggc

SEQ ID NO: 3 - DNA, 3915 bp, Cairina moschata
atggcgggcgcggagcccttcggcgccgtgctgggcgccctgcgggactgctacgcgcaggcggccccgctggagaccttcctccgggggctgggggagagcggcgccgaggaagccgaggtggtgcgggacgacgacgccgcctgctaccgcaccttcgtgtcccagtgcgtggtgtgtgtcccccacggcgcccgcgacatcccccggcccttcagcttggagcagttatctagtcagagcgaagtcatctcaagagtcatgcagaggctgtgtgggaaaaagaagaagaacatcctcacatatggatactccttgctggatgaaaacagttctcacttccaaatcatgccgctctcaaacgtgtacagctacctgcccaacaccgcaacagaaaccatgcgtatcagtggcctctgggaaacgctgctgagcaggataggggatgacgtgatgatgtatttattggagcactgtgcaatctttatgctggttccccctagtaactgttaccaagtctgtgggcaaccaatttatgaacttatttcgcaaaatgtagaatcagccccagcgtttgttaaacaacggctttcaaagcacaaacgtagtagcttgcttaagtatacgcagaaaaggctaacgtttcacagacagtatctttcaaagtcacgtcagtcgaaacgcaggcaaagacttgaagctaatgtctccagcatgagaaataaaaccagcaataatatacaaagcctagggtccgctgctctggaaaaacagagtagctccaatgcaggtttgtcagctacagcaccatccttaaaaaggaagcttgctagggaacaactggaagtcacggctaagagagcaagattagaagagaaagagagggaggaacaggcttgtaatactgctcctaatgtaaaccagagtattcccaagaggtatggaaccagctgtgtagcatcacgttctgtaagtcttattaaagaaaaatacatttctcaaagaagtaacagtgatatgtctcgtccttctttagttcacaattctcatcatgggaagaagtctgtggcagacaaaagctctttcctgcaaggagctgagagtaacagacatttaaagcccagcattgaaatgcaagcaggatccagcaggaagagagtagagatacacaggcctatacctcggttggattggataccaatcgaaccggcggaaagtagttcttcaggacacaaaaagcaggaaagtcccctagctcatctggcagaggagttaccaaatagggttttgccatctacaatatacattgacaggaagtttcttctgtattctcgcaggtactggggggaacgtttcccaaaatccttcctattgaatcgcctgaagggtagtgaggcaggtgtaaagcgactaatagaaacgatattcttaagccaaaatccgtttgggcaaaagcgcaaccaaggtctgccacagaaaaaatggagaaagaagaagcttcccaaacgcttctggagaatgagaagtacgtttcaaaaactcttaaagaatcatggaaagttcccttacgtagctttcttgagacaaaattgccctcttcggatatctgaaaccattttgggaaaagccaagctgctcagtcgggcacctttgcctgggcaagcagaggctcacaagcaagcagaacagcttgggaaggagcctgctaagcgtgtggcaagcagcagatgcgaatctggtcacaccaacgtgcccagcagcgtacgcgctcctctcgcagcatctgcgtgcgtggagccagggggggaggagcagatccctgcagaggcgtctgattcagtcctcagggagcttctcaaggagcactgcagccacttccaggtgtacctctttgtgagggagtgcgtggagcgggtgatccccgccgagctctggggttcaaaccataacaagcgccggttcttcaagaacgtgaaagcattcatttccatggggaagtacgctaagctttccttgcaggtgttgatgtggaagatgagagtaaatgactgcatgtggcttcgtctggccaaaggtaatcactttgttcctgcctctgaacaccgttaccgtgaagaaattttggctaaattcctatactggctgatggatacgtatgttgttgagttgctcagatcatttttctatatcaccgagaccatgttccagaaaaatatgcttttctactaccgaaagtgtatttgggccaagttacaggacattggaattagaaagcattttgccaaagtacagctacgtcctttaactgcagaggagatggaagcgatccatcagaaaaaataccttcctatggcatcaaagctccgtttcattcccaaagtcagtggactaagacccatcgtcagaatgagcggtgttgttgaagcacaaacgttgagcaaggaaagcagagcaaagaagatgaatcactacaacatgcaactgaaaaatctatttagtgtgttaaattatgaacgaactataaacaccagttacatcggctcttcagtgtttgggagagatgatatctacaagaagtggaagacatttgttaaaaaggttcttaaatcagatggtgaaattcctcatttctactatgtaaaggccgatgtgtccagggcttttgatagcattcctcacgataaacttgtggaagtgatttcacaggtcttaaaacctgagaaaaaaactgtctactgcatacggcgctatgcagtggttatgatcactggaagtggaaaaaccaggaagttatacaggagacatgtttctactttcaaggattttatgccagacatgaagcagtttgtgtcccggcttcatgagagtacctcattgcgagatgcaataatagttgaacagagcttaactttcaatgagacaagtgccagtctatttaatttttttcttcaaatgctaaataataacatcctggaaattgagcgcagctactacttacagtgctctggaattccacagggctcccttttgtcaaccttgctttgcagcttgtgctatggagacatggaaaacaaattattcagtggggtacagaaggatggagtcctgatccgtctcattgatgactttttgcttgttacaccacacttaacgcatgcaagaactttcctaaggactctagcaatgggcattcctgagtatggctttttgataaaccccaaaaagacggtggtgaatttttctgttgacgatatcccagagtgttccgaatttaaacagctgccaaactgtcgtttgatcccatggtgtggcttattattggatacacagacacttgaggtttactgtgattactccagctattcctgtacttctatcagatcaagtctttccttcaattcaaacagaacagctgggaaaaacatgaaacacaaattggttgcagtccttaaactgaaatgccatggcttgtttcttgatttacagatcaatagcgttaaaacagttttcattaatgtctacaagatatttttacttcaggcttacaggttccatgcctgtgttattcaacttccattcaaccagaaagttaggaacaatcctgatttcttcctcagagtcatcgctgagaatgcatcgtgctgctattctatgctgaaagctaaaaatccagggtttactttaggtaacagaggtgcatctggcatgttcccttctgaggcagcagagtggctctgctatcatgccttcactgtcaaactgtcaaaccacaaagttgtttacaaatgcttgcttaagcccctgaagttctgtatgacacagctattccggaagatcccaaaggatactaaggcactactgaagacagtgacagaaccatctatttgtcaagatttcaaagctatcctggactga

(19) United States
(12) Patent Application Publication
(10) Pub. No.: US 2013/0095557 A1
(43) Pub. Date: Apr. 18, 2013

(54) IMMORTALIZED AVIAN CELL LINES

(71) Applicants: Philippe Erbs, Dagneux (FR); Marina Kapfer, Castelnau-d'Etretefonds (FR); Nathalie Silvestre, Ergersheim (FR)

(72) Inventors: Philippe Erbs, Dagneux (FR); Marina Kapfer, Castelnau-d'Etretefonds (FR); Nathalie Silvestre, Ergersheim (FR)

(73) Assignee: TRANSGENE S.A., Illkirch Graffenstaden (FR)

(21) Appl. No.: 13/806,428

(22) PCT Filed: Jun. 29, 2011
(86) PCT No.: PCT/EP2011/060952

§ 371 Date: Dec. 21, 2012

Related U.S. Application Data
(63) Continuation-in-part of application No. 12/829,773, filed on Jul. 2, 2010, now Pat. No. 8,357,531.

(51) Int. Cl. C12N 5/073

(52) U.S. Cl. CPC C12N 5/0603
USPC 435/235.1; 435/349

(57) ABSTRACT

This invention relates to immortalized avian cells, including those deposited under accession numbers 09070701, 09070702, and 09070703 at the ECACC, and to the use of these cells for the production of viruses. The cells according to the invention are particularly useful for the production of recombinant viral vectors which can be used for the preparation of therapeutic and/or prophylactic compositions for the treatment of animals and more particularly humans.

### BACKGROUND OF THE INVENTION

1. Technical Field of the Invention

This invention relates to immortalized avian cells, and to the use of these cells for the production of viruses. The cells according to the invention are particularly useful for the production of recombinant viral vectors which can be used for the preparation of therapeutic and/or prophylactic compositions for the treatment of animals and more particularly humans.

2. Description of Background and/or Related Art

Eukaryotic cell lines are fundamental for the manufacture of viral vaccines and many products of biotechnology. Biologicals produced in cell cultures include enzymes, hormones, immunobiologicals (monoclonal antibodies, interleukins, lymphokines), and anticancer agents. Although many simpler proteins can be produced using bacterial cells, more complex proteins that are glycosylated, currently must be made in eukaryotic cells.

Avian cells have been used for years for the production of viral vectors. For example, the Vaccinia virus used for preparing prophylactic composition for the treatment of Variola was cultivated on Chicken Embryonic Fibroblast (CEF). Avian cells are particularly useful since many virus used in pharmaceutical composition are able to replicate on them. More noticeably, various viruses are only able to grow on avian cells. This is for example the case of Mammalian Virus Ankara (MVA) which is unable to grow on mammalian cells. This poxvirus, which derived from a Vaccinia Virus by more than 500 passages on CEF, was used in the early seventies for vaccinating immunodeficient peoples against Variola. Now, MVA is mainly used as a vector for gene therapy purposes. For example, MVA is used as a vector for the MUC1 gene for vaccinating patients against tumor expressing this antigen (Scholl et al., 3 J BIOMED BIOTECHNOL. 194-201 (2003)). MVA carrying the gene coding HPV antigens are also used as a vector for the therapeutic treatment of ovarian carcinoma. More recently, MVA has been the vector of choice for preparing prophylactic treatment against newly emerging diseases or probable biological weapons such as west nile virus and anthrax.

With this respect, there is a growing need for virus production. For now, the most used MVA production process comprises a virus replication step on CEF. However the use of CEF is linked to various difficulties. Firstly, the preparation of CEF comprised many steps which have to be done manually.

Furthermore, this virus production process depends on the availability of eggs which may be totally disrupted in case of contamination of the breedings. This problem is more and more relevant with the spread of Avian Flu.

Additionally, many CEFs possess a reverse transcriptase activity (RT). RT is an enzyme necessary for retroviruses to reproduce. Retroviruses are found in many different species. RT is not infectious in humans or animals, and it has not been shown to cause any adverse health effects in people. Using a highly sensitive polymerase chain reaction (PCR) based assay, RT activity has been detected in minute quantities in vaccines manufactured with chick embryo fibroblasts. The source of the enzyme is probably a partial viral genome coding for RT, believed to be integrated into chick cells hundreds or thousands of years ago. Avian retroviruses that produce this RT are not known to affect humans. While the human immunodeficiency virus (HIV, the virus that leads to AIDS), is a retrovirus, the RT activity detected in vaccines is definitively not derived from HIV. Furthermore, the presence of RT does not confirm the presence of a retrovirus. Nevertheless, a cell line with no endogenous RT activity would be of interest.

In order to emancipate virus production process from the use of CEF, there is an increasing need for an avian cell line which would allow the replication and the production of the virus. Immortalized cell lines can be maintained or frozen from batch to batch on the production site and are always available for a new production process. Moreover as they are confined at the production plant, they are less subject to contamination by exogenous contaminant. Their use allows a drastic reduction of the manual manipulation needed for the production process. All these properties lead to a reduction of the price and of the duration of the production process as well as a diminution of the potential contamination.

Finally, cell lines can be fully characterized and are thus totally compliant with the good laboratory practice and the requirements of the different medical agencies. Different avian cell lines have already been described. For example, DF1 (U.S. Pat. No. 5,879,924) is a spontaneously immortalized chicken cell line derived from 10 day old East Lansing Line (ELL-0) eggs. The cells are useful as substrates for virus propagation, recombinant protein expression and recombinant virus production. However, this cell line is susceptible to various virus such as Meleagrid herpesvirus 1 (Herpes Virus of Turkey), Fowlpox Virus, reovirus, Avian Sarcoma Leukemia Virus and Rous Sarcoma Virus. Immortal avian cells can also be derived from embryonic stem cells by progressive severance from growth factors and feeder layer, thus maintaining growth features and infinite lifespan characteristic of undifferentiated stem). The only available avian cell line derived by this process is the Ebx chicken cell line (WO 2005/007840) which has been in contact with feeder layers from murin origin, raising additional regulatory questions like murin virus contamination and presence of endogenous retroviral sequences in chicken cells. Moreover this cell lines have been described in some conditions as unstable and differentiation-prone.

A duck embryo permanent cell line, free from endogenous avian retroviruses has also been established. The cell line, designated as DEC 99 (Ivanov et al. EXPERIMENTAL PATHOLOGY AND PARASITOLOGY, 4/2000 Bulgarian Academy of Sciences) has been cultured over 140 consecutive passages and it is not tumorigenic for birds. The DEC 99 cell line is a standard cell culture system that has been used for research and can be applied for the needs of biotechnology. This cell line is a suitable model for studies in the field of cell biology, virology, immunology, toxicology and for the production of diagnostics and vaccines. The susceptibility of the permanent duck embryo cell line (CL) DEC 99 to infection with embryo-adapted avian poxvirus (APV) vaccine strains have been studied (Ivanov et al. EXPERIMENTAL PATHOLOGY AND PARASITOLOGY, 4/6 2001 Bulgarian Academy of Sciences). The FK and Dessau vaccine strains of fowl and pigeon origin respectively have been used. The virus strains were consecutively passaged (13 passages) on primary duck embryo cell cultures (CCs). The adapted virus strains have been further passaged (12 passages) in the CCs of the DEC 99 cell line, where a typical cytopathic effect (CPE) was observed. The production of infectious virions was checked by inoculation of 11-day-old White Leghorn embryos, where typical pox proliferations on the chorioalantoic membranes (CAMs) were formed. In the DEC 99 cells the FK strain caused early CPE, compared to the Dessau strain and reached a titer of 106,25 CClD50/ml. The DEC 99-adapted virus strains induced typical cutaneous “takes” after vaccination of two-month-old chicks. Thus, the DEC 99, as a standard CC system appears to be suitable for production of vaccines against fowl pox. Nevertheless this particular cell line is slow growing after passage 40 and is unable to grow in suspension.

Nucleic acid sequences from the Early region of human Adenovirus 5 have already been used to transform some specific human cells in vitro (293 and PER. C6 cell lines; Fallaux, F. J. et al., 9 HUM. GENE THER. 1909-17 (1998); Graham, F. L. et al., 36 J. GEN. VIROL. 59-74 (1977)).

In general terms, the adenoviral genome consists of a double-stranded linear DNA molecule approximately 36 kb in length which contains the sequences coding for more than 30 proteins. At each of its ends, a short inverted sequence of 100 to 150 nucleotides, depending on the serotypes, designated ITR (inverted terminal repeat), is present. ITRs are involved in the replication of the adenoviral genome. The encapsidation region of approximately 300 nucleotides is located at the 5′ end of the genome immediately after the 5′ ITR.

The early genes are distributed in 4 regions which are dispersed in the adenoviral genome, designated E1 to E4 (E denoting “early”). The early regions comprise at least six transcription units which possess their own promoters. The expression of the early genes is itself regulated, some genes being expressed before others. Three regions, E1, E2 and E4, respectively, are essential to the viral replication. Thus, if an adenovirus is defective for one of these functions, that is to say if it cannot produce at least one protein encoded by one of these regions, this protein will have to be supplied to it in trans.

The E1 early region is located at the 5′ end of the adenoviral genome, and contains 2 viral transcription units, E1A and E1B, respectively. This region codes for proteins which participate very early in the viral cycle and are essential to the expression of almost all the other genes of the adenovirus. In particular, the E1A transcription unit codes for a protein which trans-activates the transcription of the other viral genes, inducing transcription from the promoters of the E1B, E2A, E2B and E4 regions.

It was shown by Guilhot et al. (Guilhot, C. et al., 8 ONCOGENE 619-24 (1993)) that retroviral transduction of the 12S protein of E1A from Ad5 can lead to immortalization of quail cells. However, WO 2005/042728 disclosed that it is impossible to immortalize avian cells when the E1A gene is introduced by transfection of naked DNA instead of retrovirus infection. WO 2005/042728 further states: “that the extremely efficient and stable transduction via retrovirus infection creates a cell pool large enough to harbor individual cells with spontaneous genomic changes that have blocked apoptosis that normally is induced upon Retinoblastoma inactivation.” (page 10).

The presence of retroviral sequences in the cells obtained by Guilhot et al. hinder the use of such cells for the production of biological product and more particularly for therapeutic compounds.

### BRIEF SUMMARY OF THE INVENTION

The inventors have surprisingly found that avian cells, and more particularly cairina moschata cells, can be efficiently immortalized by E1A transfection with a non-viral vector. In order to solve the different problems linked to the use of CEF and/or to the use of previously available cell lines, the present invention provides an immortalized avian cell comprising an E1A nucleic acid sequence characterized in that said cell is obtained by a process comprising the step of transfecting the cell with a non viral vector comprising said E1A nucleic acid sequence and wherein said cell does not comprise an E1B nucleic acid sequence.

The present invention also refers to a process for immortalizing an avian cell comprising the step of transfecting said cell with a non-viral vector comprising an E1A nucleic acid sequence and wherein said process does not comprise a step of transfecting said cell with an E1B nucleic acid sequence.

The present invention includes immortalized avian cell lines deposited at the European Collection of Cell Cultures (ECACC) under accession numbers 09070701, 09070702, and 09070703 and derivatives thereof. The immortalized avian cells may further comprise one or more nucleic acid sequences that allow propagation of a defective virus. The immortalized avian cells may further comprise a nucleic acid coding a substance of interest. The immortalized avian cell lines may be used for the replication of a virus, including live, attenuated, or recombinant viruses. The virus to be replicated using the immortalized avian cell lines may be poxvirus (such as vaccinia virus, e.g., a modified vaccinia virus Ankara (MVA)), adenovirus, retrovirus, herpervirus, alphavirus, foamy virus, adenovirus-associated virus, flavivirus and influenza virus.

Other characteristics, aspects, and advantages of the invention are set forth in the detailed description that follows.

### DETAILED DESCRIPTION OF THE FIGURES

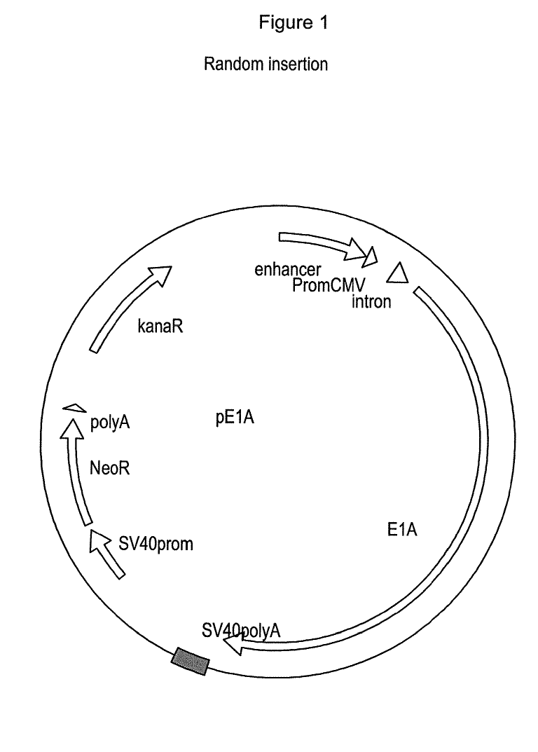

FIG. 1: Vector comprising a gene coding the E1A nucleic acid sequence.

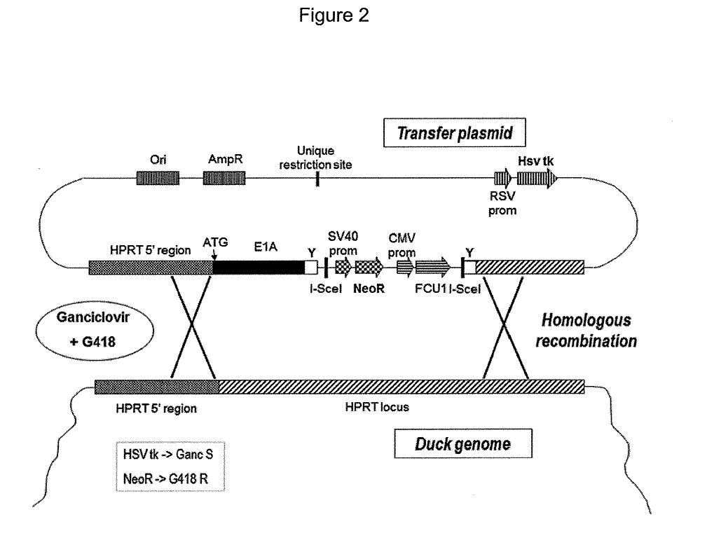

FIG. 2: Schematic representation of the site specific insertion of the E1A nucleic acid sequence into the HPRT gene.

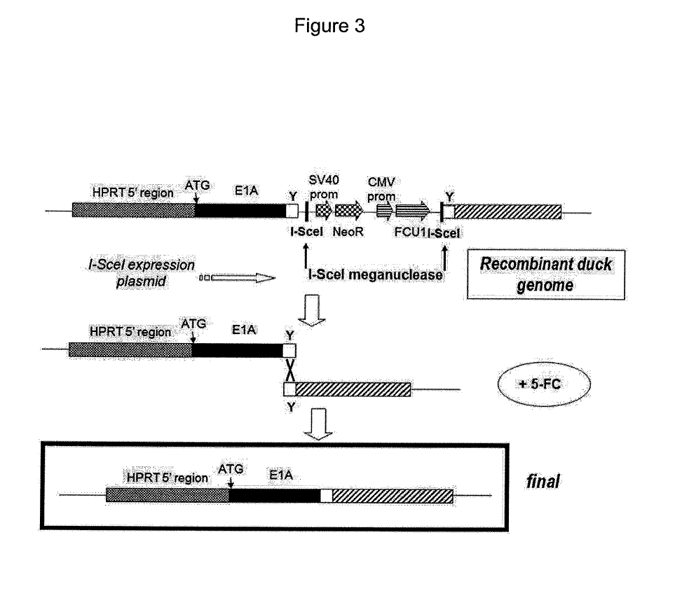

FIG. 3: Schematic representation of the elimination of the first and the third selection marker from the genome of the immortalized cell obtained by the process of the invention.

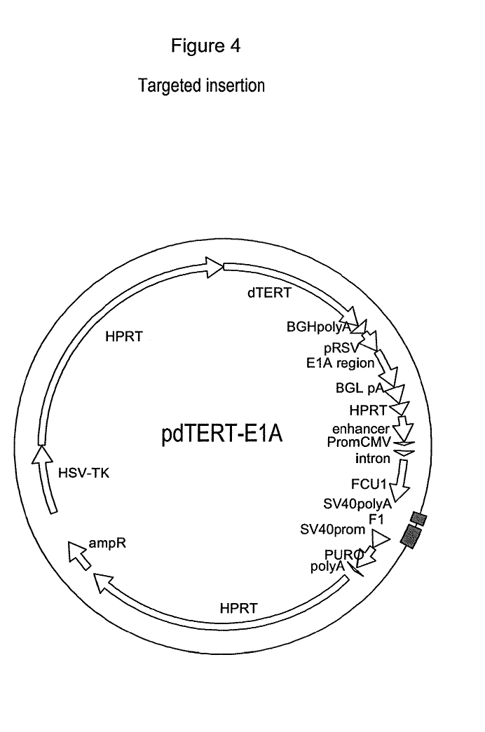

FIG. 4: Vector comprising a gene coding the Cairina moschata telomerase reverse transcriptase gene and the E1A nucleic acid sequence.

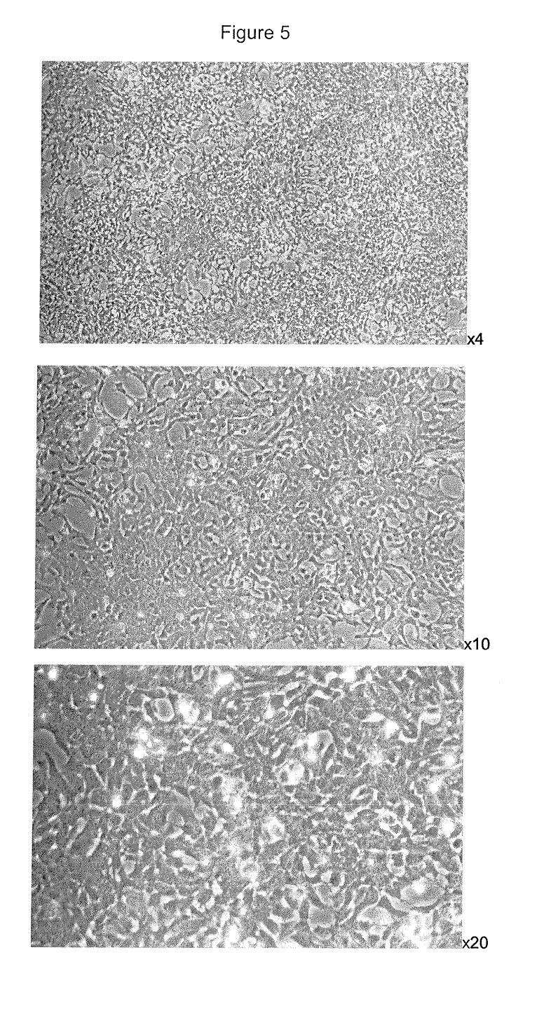

FIG. 5: Light microscopy imaging of the Cairina moschata immortalized avian cell line ECACC 09070701 (passage 21).

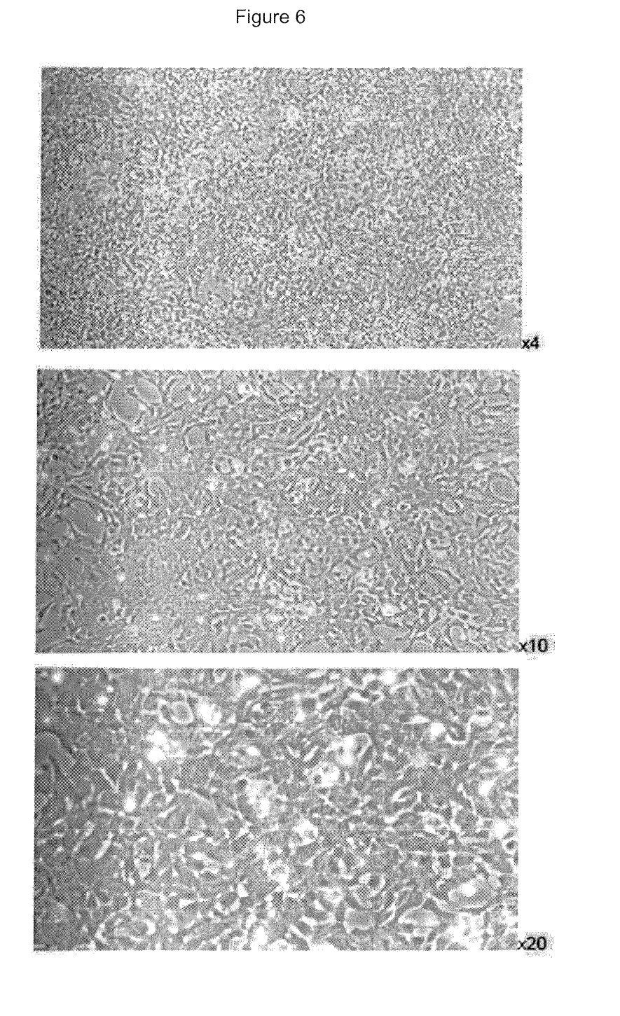

FIG. 6: Light microscopy imaging of the Cairina moschata immortalized avian cell line ECACC 09070702 (passage 24).

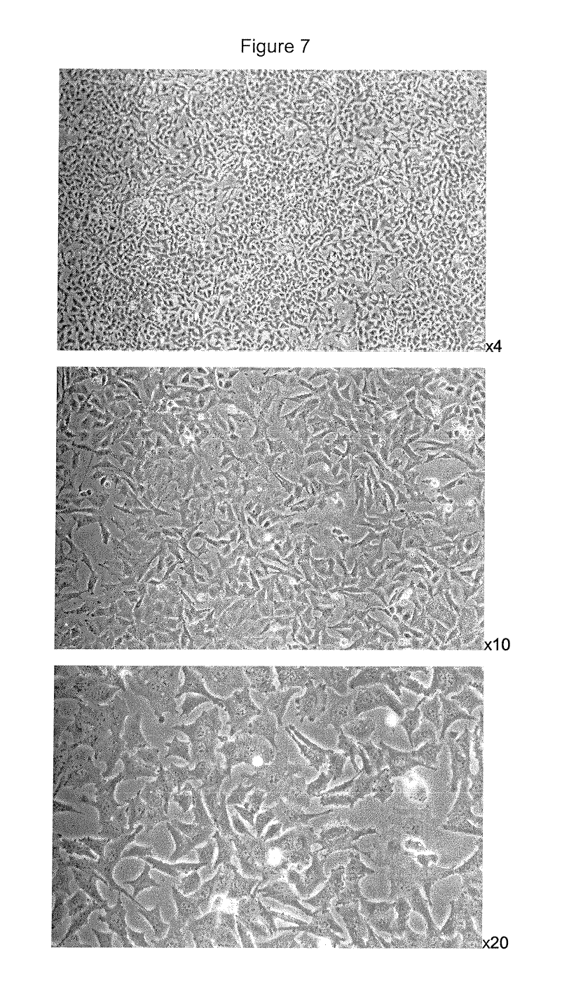

FIG. 7: Light microscopy imaging of the Cairina moschata immortalized avian cell line ECACC 09070703 (passage 28).

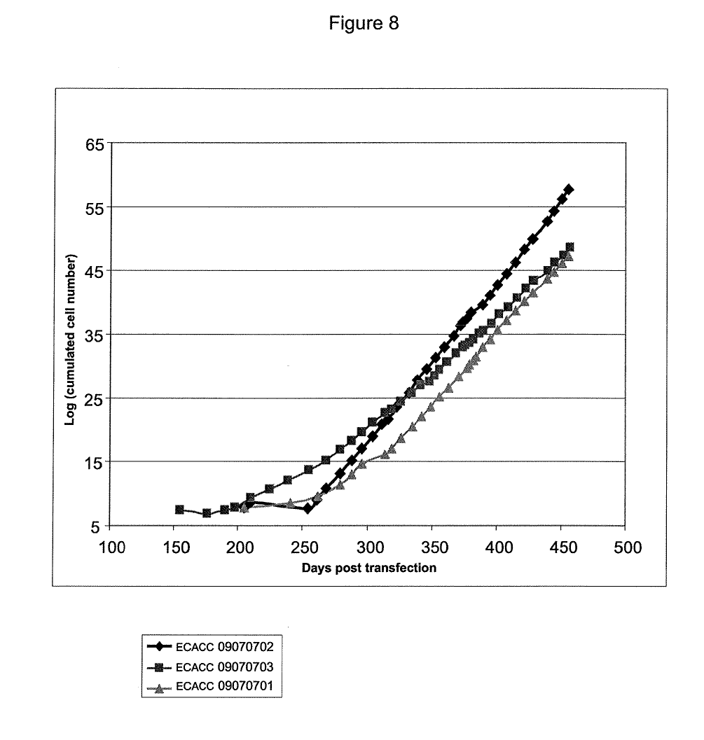

FIG. 8: Cairina moschata immortalized avian cell line ECACC 09070701, ECACC 09070702, and ECACC 09070703 growth curve.

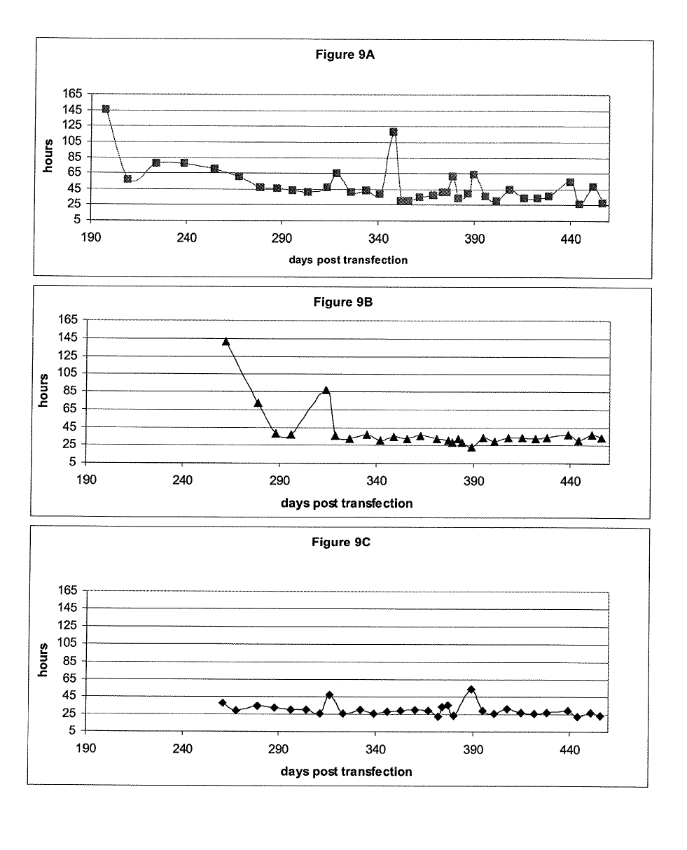

FIG. 9: Cairina moschata immortalized avian cell line ECACC 09070703 (FIG. 9A), ECACC 09070701 (FIG. 9B), and ECACC 09070702 (FIG. 9C) population doubling time evolution.

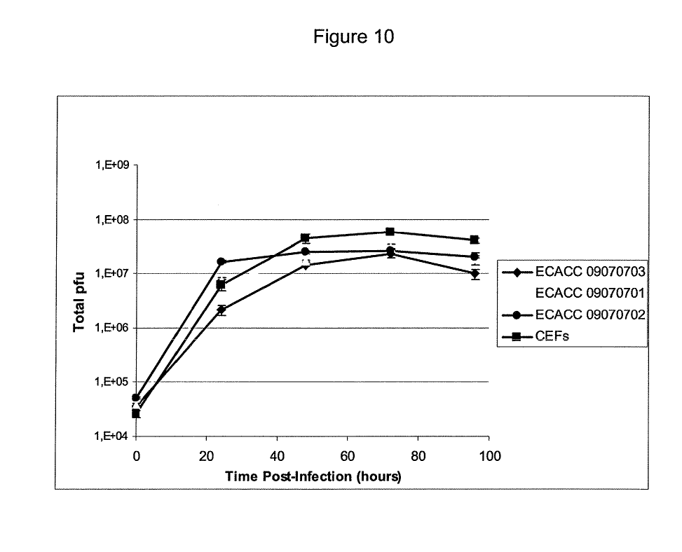

FIG. 10: Production of Modified Vaccinia virus Ankara (MVA) in the Cairina moschata immortalized avian cell line ECACC 09070701, ECACC 09070702, and ECACC 09070703 (MOI 0,05).

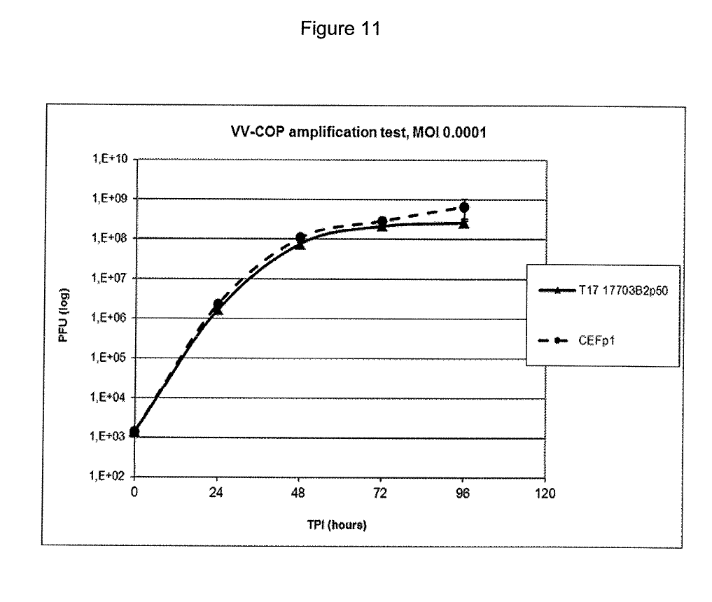

FIG. 11: Production of VV-COP in the Cairina moschata immortalized avian cell line ECACC 09070702

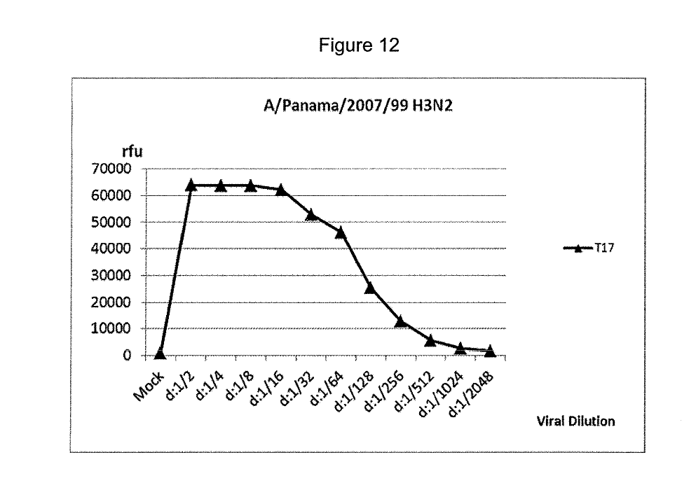

FIG. 12: Production of influenza A/Panama/2007/99 H3N2 virus strain in the Cairina moschata immortalized avian cell line ECACC 09070702

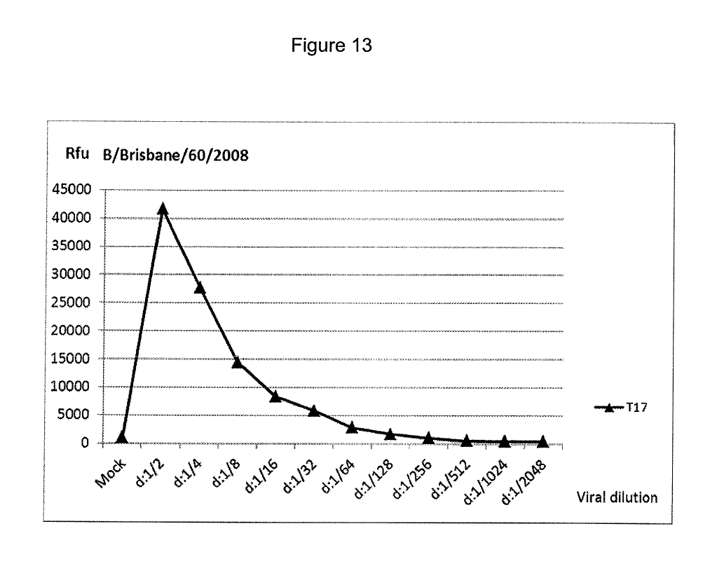

FIG. 13: Production of influenza B/Brisbane/60/2008 virus in the Cairina moschata immortalized avian cell line ECACC 09070702.

### DETAILED DESCRIPTION OF THE INVENTION

The inventors have surprisingly found that avian cells, and more particularly cairina moschata cells, can be efficiently immortalized by E1A transfection with a non-viral vector.

In order to solve the different problems linked to the use of CEF and/or to the use of previously available cell lines, the present invention provides an immortalized avian cell comprising an E1A nucleic acid sequence characterized in that said cell is obtained by a process comprising the step of transfecting the cell with a non viral vector comprising said E1A nucleic acid sequence and wherein said cell does not comprise an E1B nucleic acid sequence.

The present invention also refers to a process for immortalizing an avian cell comprising the step of transfecting said cell with a non-viral vector comprising an E1A nucleic acid sequence and wherein said process does not comprise a step of transfecting said cell with an E1B nucleic acid sequence.

The present invention includes immortalized avian cell lines deposited at the European Collection of Cell Cultures (ECACC) under accession numbers 09070701, 09070702, and 09070703 and derivatives thereof. The immortalized avian cells may further comprise one or more nucleic acid sequences that allow propagation of a defective virus. The immortalized avian cells may further comprise a nucleic acid coding a substance of interest. The immortalized avian cell lines may be used for the replication of a virus, including live, attenuated, or recombinant viruses. The virus to be replicated using the immortalized avian cell lines may be poxvirus (such as vaccinia virus, e.g., a modified vaccinia virus Ankara (MVA)), adenovirus, retrovirus, herpervirus, alphavirus, foamy virus, adenovirus-associated virus, flavivirus and influenza virus.

An immortalized cell, as used herein, refers to a cell capable of growing in culture for more than 35 passages.

The term passage number refers to the number of times that a cell population has been removed from the culture vessel and undergone a subculture (passage) process, in order to keep the cells at a sufficiently low density to stimulate further growth.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or”, and “all or any other combination of the elements connected by said term”.

As used herein, the term “comprising” is intended to mean that the products, compositions and methods include the referenced components or steps, but not excluding others. “Consisting essentially of” when used to define products, compositions, and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, the term “E1A nucleic acid sequence” refers to nucleic acid sequence coding all gene products of the adenovirus E1A region, including the nucleic acid sequence coding the two major RNAs: 13S and 12S.

Preferably, the term “E1A nucleic acid sequence” refers to a nucleic acid sequence comprising a nucleic acid sequence which has at least 60% sequence identity to SEQ ID NO:1. In a more preferred embodiment of the invention, E1A refers to a nucleic acid sequence comprising a nucleic acid sequence which has at least 70%, preferably at least 80%, and even more preferably at least 90%, nucleic acid sequence identity to SEQ ID NO:1. In a more preferred embodiment, E1A refers to the nucleic acid sequence set forth in SEQ ID NO:1.

As used herein, the term “E1B nucleic acid sequence” refers to all nucleic acid sequence of the adenovirus E1B region, including the nucleic acid sequence coding the 3 major polypeptides, of 19 kd and 55 kd.

As employed herein, the term “substantially the same nucleic acid sequence” refers to nucleic acid molecule having sufficient identity to the reference polynucleotide, such that it will hybridize to the reference nucleotide under moderately stringent hybridization conditions. In one embodiment, nucleic acid molecule having substantially the same nucleotide sequence as the reference nucleotide sequence set forth in SEQ ID NO:1.

Hybridization refers to the binding of complementary strands of nucleic acid (i.e., sense:antisense strands or probe:target-DNA) to each other through hydrogen bonds, similar to the bonds that naturally occur in chromosomal DNA. Stringency levels used to hybridize a given probe with target-DNA can be readily varied by those of skill in the art.

The phrase “stringent hybridization” is used herein to refer to conditions under which polynucleic acid hybrids are stable. As known to those of skill in the art, the stability of hybrids is reflected in the melting temperature (Tm) of the hybrids. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5*Denhart's solution, 5*SSPE, 0.2% SDS at 42° C., followed by washing in 0.2*SSPE, 0.2% SDS, at 65° C.

As used herein, the expression “non-viral vector” notably refers to a vector of plasmid origin, and optionally such a vector combined with one or more substances improving the transfectional efficiency and/or the stability of said vector and/or the protection of said vector in vivo toward the immune system of the host organism. These substances are widely documented in the literature which is accessible to persons skilled in the art (see, e.g., Feigner et al., 32 PROC. WEST. PHARMACOL. SOC. 115-121 (1987); Hodgson and Solaiman, 14 NATURE BIOTECHNOLOGY 339-342 (1996); Remy et al., 5 BIOCONJUGATE CHEMISTRY 647-654 (1994)). By way of illustration, but without limitation, they may be polymers, lipids, in particular cationic lipids, liposomes, nuclear proteins, or neutral lipids. These substances may be used alone or in combination. Examples of such compounds are in particular available in WO 98/08489, WO 98/17693, WO 98/34910, WO 98/37916, WO 98/53853, EP 890362, or WO 99/05183. A combination which may be envisaged is a plasmid recombinant vector combined with cationic lipids (DOGS, DC-CHOL, spermine-chol, spermidine-chol and the like) and neutral lipids (DOPE).

The choice of the plasmids which can be used in the context of the present invention is vast. They may be cloning and/or expression vectors. In general, they are known to a person skilled in the art and a number of them are commercially available, but it is also possible to construct them or to modify them by genetic engineering techniques. There may be mentioned, by way of examples, the plasmids derived from pBR322 (Gibco BRL), pUC (Gibco BRL), pBluescript (Stratagene), pREP4, pCEP4 (Invitrogene) or p Poly (Lathe et al., 1987, Gene 57, 193-201). Preferably, a plasmid used in the context of the present invention contains a replication origin ensuring the initiation of replication in a producing cell and/or a host cell (for example, the CoIE1 origin may be selected for a plasmid intended to be produced in E. coli and the oriP/EBNA1 system may be selected if it is desired for it to be self-replicating in a mammalian host cell, Lupton and Levine, 5 MOL. CELL. BIOL. 2533-2542 (1985); Yates et al., 313 NATURE 812-815 (1985). It may comprise additional elements improving its maintenance and/or its stability in a given cell (cer sequence which promotes the monomeric maintenance of a plasmid (Summers and Sherrat, 36 CELL 1097-1103 (1984), sequences for integration into the cell genome)).

The term “non-viral vector” excludes viral vectors, such as, for example vector deriving from a poxvirus (vaccinia virus, in particular MVA, canarypox and the like), from an adenovirus, from a retrovirus, from a herpesvirus, from an alphavirus, from a foamy virus, from an adeno-associated virusfrom a flavivirus or from an influenza virus.

The present invention also relates to cells deriving from the cell according to the invention. As used herein, the term “derived” refers to cells which develop or differentiate from or have as ancestor a cell according to the invention.

The term passage number refers to the number of times that a cell population has been removed from the culture vessel and undergone a subculture (passage) process, in order to keep the cells at a sufficiently low density to stimulate further growth.

As used herein, the term “transfected” refers to the stable transfection or the transient transfection of the cell of the invention.

The term “stable transfection” or “stably transfected” refers to the introduction and integration of foreign nucleic acid sequence into the genome of the transfected cell. The term “stable transfectant” refers to a cell that has stably integrated foreign DNA into the genomic DNA.

According to a preferred embodiment of the invention, the avian cell of the invention derives from a cell of the Anatidae family or of the Phasianidae family. Among Anatidae, cells belonging to the Cairina or Anas genus are particularly preferred. Even more preferably, the cells according to the invention belong to the Cairina moschata or to the Anas platyrhynchos species.

Preferably, the cell according to the invention is taken from an embryonic organism. Methods allowing the isolation of cells from a living organism are well known to the one skilled in the art. For example, methods disclosed in Example 2 can be used. According to a preferred embodiment of the invention, the primary cell is isolated from an embryo belonging to the Anatidae family which is between 0 and 20 days old, more preferably between 5 and 15 days old, and even more preferably between 11 and 14 days old.

According to a preferred embodiment of the invention, the E1A nucleic acid sequence is inserted into a target DNA sequence of the cell according to the invention.

As used herein, a “target DNA sequence” is a predetermined region within the genome of a cell which is targeted for modification by homologous recombination with the vector. Target DNA sequences include structural genes (i.e., DNA sequences encoding polypeptides including in the case of eucaryotes, introns and exons), regulatory sequences such as enhancers sequences, promoters and the like, and other regions within the genome of interest. A target DNA sequence may also be a sequence which, when targeted by a vector, has no effect on the function of the host genome.

As used herein, “inserted into a target DNA sequence” widely means that the homologous recombination process which leads to the insertion of the immortalizing gene introduces a deletion or a disruption into the targeted DNA sequence.

To produce immortalized avian cell wherein the DA nucleic acid sequence is inserted into a target DNA sequence, the vector used in the process according to the invention can further comprise two homologous sequences capable of homologous recombination with a region of a target DNA sequence native to the genome of said cell genome.

The presence of said homologous sequences allows the site specific insertion of the nucleic acid molecule of the invention into the target DNA sequence by homologous recombination.

The term “homologous recombination” refers to the exchange of DNA fragments between two DNA molecules at the site of essentially identical nucleotide sequences. According to this particular embodiment of the invention, within the vector are sequences which are homologous with sequence portions contained within the target DNA sequence. In a preferred embodiment of the invention, the homologous sequences in the transfer vector are hundred percent homologous to the region of the target sequence. However, lower sequence homology can be used. Thus, sequence homology as low as about 80% can be used.

The homologous sequences in the transfer vector comprise at least 25 bp. Longer regions are preferred, at least 500 bp, and more preferably at least 5,000 bp.

According to a more preferred embodiment of the invention, the nucleic acid molecule is surrounded by the homologous sequences in the vector.

As used herein “surrounded” means that one of the homologous sequences is located upstream of the nucleic acid molecule of the invention and that one of the homologous sequences is located downstream of the nucleic acid molecule of the invention. As used herein, “surrounded” does not necessarily mean that the two homologous sequences are directly linked to the 3′ or to the 5′ end of the immortalizing gene. The immortalizing gene and the homologous sequences can be separated by an unlimited number of nucleotides.

One skilled in the art is able to choose the appropriate homologous sequences in order to target a specific DNA sequence into the genome of the cell to be immortalized. For example, one homologous sequence can be homologous to a part of the targeted sequence, wherein the other homologous sequence is homologous to a DNA sequence located upstream or downstream of the targeted sequence. According to another example, one of the homologous sequences can be homologous to a DNA sequence located upstream of the targeted DNA sequence, wherein the other homologous sequence is homologous to a DNA sequence located downstream of the target DNA sequence. In another example, both the homologous sequences are homologous to sequences located into the target DNA sequence.

According to a preferred embodiment of the invention, the target DNA sequence is the HPRT (Hypoxanthine phosphorybosyl transferase) gene.

The genomic sequence comprising the HPRT promoter and the HPRT gene of cairina moschata is set forth in SEQ ID NO:2. The sequence coding the HPRT starts at the ATG codon in position 8695 of the nucleic acid sequence set forth in SEQ ID NO:2, the sequence upstream of this ATG codon is the HPRT promoter sequence.

One skilled in the art is able to choose the homologous sequences necessary for the integration of the E1A nucleic acid sequence into the HPRT gene. As between the various members of a family, the genomic sequences coding HPRT are highly homologous. One skilled in the art is also able to design the homologous sequences necessary to target the HPRT gene of every avian cells.

According to a more preferred embodiment of the invention, the homologous sequences are customized in order to insert the E1A nucleic acid sequence downstream of the cell's HPRT promoter. In this particular embodiment, the nucleic acid molecule of the invention is operably linked to the cell's endogenous HPRT promoter. “Operably linked” is intended to mean that the E1A nucleic acid sequence is linked to the promoter in a manner which allows for its expression in the cell.

According to this particular embodiment, the homologous sequence, upstream of the nucleic acid molecule of the invention, has preferably a nucleic acid sequence which is homologous with at least 500 contiguous by and more preferably at least 5,000 contiguous by of the nucleic acid sequence starting from the nucleotide at position 1 and ending with the nucleotide at position 8694 of the nucleic acid sequence set forth in SEQ ID NO:2, with the proviso that said homologous sequence is not homologous with the nucleic acid sequence starting with the nucleotide at position 8695 and ending with the nucleotide at position 26916 of the nucleic acid sequence set forth in SEQ ID NO:2. Moreover, this upstream homologous sequence is preferably directly linked to the start codon of the E1A nucleic acid sequence. According to an even more preferred embodiment of the invention, the homologous sequence upstream the nucleic acid molecule of the invention consists in the nucleic acid sequence starting from the nucleotide at position 1 and ending with the nucleotide at position 8694 of the nucleic acid sequence set forth in SEQ ID NO:2. The homologous sequence, downstream of the E1A nucleic acid sequence, preferably has a nucleic acid sequence which is homologous with at least 500 contiguous by and more preferably at least 5000 contiguous by of the nucleic acid sequence starting from the nucleotide at position 10580 and ending with the nucleotide at position 18009 of the nucleic acid sequence set forth in SEQ ID NO:2. And, more preferably, said homologous sequence, downstream of the E1A nucleic acid sequence, consists in the nucleic acid sequence starting from the nucleotide at position 10580 and ending with the nucleotide at position 18009 of the nucleic acid sequence set forth in SEQ ID NO:2.

Accordingly, the present invention also relates to an avian cell comprising an E1A nucleic acid sequence characterized in that said cell is obtained by a process comprising the step of transfecting the cell with a non viral vector comprising said E1A nucleic acid sequence, wherein said cell does not comprise an E1B nucleic acid sequence and wherein said E1A nucleic acid sequence is operably linked to the cell's endogenous HPRT promoter.

According to a preferred embodiment, the vector used in the process according to the invention comprises a first selection marker, wherein this first selection marker is a positive selection marker and wherein said first selection marker is surrounded by the homologous sequences comprised in the vector. With this respect, the homologous recombination process which occurs between the vector and the genome of the cell leads to the integration of the E1A nucleic acid sequence and of the first selection marker. When the transfer vector is circular, “surrounded” means that the first selection marker and the E1A nucleic acid sequence are positioned in the same section of the vector, said section being delimited by the homologous sequences.

As used herein, the term positive selection marker notably refers to a gene encoding a product that enables only the cells that carry the gene to survive and/or grow under certain conditions. Typical selection markers encode proteins that confer resistance to antibiotics or other toxins, e.g., ampicillin, neomycin, methotrexate, or tetracycline, complement auxotrophic deficiencies, or supply critical nutrients not available from complex media. In a preferred embodiment according to the invention, the first selection marker encodes a protein that confers resistance to antibiotics.

The integration of the first selection marker allows the selection of the cells that have incorporated the E1A nucleic acid sequence. Accordingly, the process according to the invention can further comprise a step wherein said cells are cultivated in a medium which only allows the growth of the cells which have incorporated the first selection marker. For example, in a medium which comprises an antibiotic.

According to a more preferred embodiment of the invention, the first selection marker, in the vector, is surrounded by sequences allowing its suppression. Said sequences allowing the suppression of the first selection marker do not surround the E1A nucleic acid sequence. When the vector is circular, the sequences allowing the suppression of the first selection marker, the first selection marker and the E1A nucleic acid sequence are positioned in the same section of the transfer vector, said section being delimited by the homologous sequences.

Sequences allowing the suppression of a nucleic acid fragment are well known to the one skilled in the art (Nunes-Duby, S. et al., 26 NUCLEIC ACIDS RES. 391-406 (1998)). These sequences can be recognized by one or more specific enzymes which induce the suppression of the nucleic acid comprised between said sequences, these enzymes are called “recombinase”. For example, three well-known recombinases allowing the suppression of a nucleic acid fragment are the FLP, ISCE1, and Cre recombinases.

A typical site-specific recombinase is Cre recombinase. Cre is a 38-kDa product of the cre (cyclization recombination) gene of bacteriophage P1 and is a site-specific DNA recombinase of the Int family. Sternberg, N. et al., 187 J. MOL. BIOL. 197-212 (1986). Cre recognizes a 34-bp site on the P1 genome called loxP (locus of X-over of P1) and efficiently catalyzes reciprocal conservative DNA recombination between pairs of loxP sites. The loxP site consists of two 13-bp inverted repeats flanking an 8-bp nonpalindromic core region. Cre-mediated recombination between two directly repeated loxP sites results in excision of DNA between them as a covalently closed circle. Cre-mediated recombination between pairs of loxP sites in inverted orientation will result in inversion of the intervening DNA rather than excision. Breaking and joining of DNA is confined to discrete positions within the core region and proceeds on strand at a time by way of transient phophotyrosine DNA-protein linkage with the enzyme.

Another site-specific recombinase is the I-SceI. Other intron-homing endonuclease, for instance I-TliI, I-CeuI, I-CreI, I-PpoI, and PI-PspI, can also be substituted for I-SceI in the process according to the invention. Many are listed by Belfort and Roberts (25 NUCLEIC ACIDS RESEARCH 3379-3388 (1997)). Many of these endonucleases derive from organelle genomes in which the codon usage differs from the standard nuclear codon usage. To use such genes for nuclear expression of their endonucleases it may be necessary to alter the coding sequence to match that of nuclear genes. I-SceI is a double-stranded endonuclease that cleaves DNA within its recognition site. I-SceI generates a 4 by staggered cut with 3′OH overhangs.

The enzyme I-SceI has a known recognition site. The recognition site of I-SceI is a non-symmetrical sequence that extends over 18 bp.

5′ TAGGGATAACAGGGTAAT3′
3′ ATCCCTATTGTCCCATTA5′

Another site-specific recombinase is the FLP recombinase. Flp recombinase recognizes a distinct 34-bp minimal site which tolerates only limited degeneracy of its recognition sequence (Jayaram, 82 PROC. NATL. ACAD. SCl. USA 5875-9 (1985); Senecoff et al., 201 J. MOL. BIOL. 405-21 (1988)). The interaction between Flp recombinase and a FRT sequence have been examined (Panigrahi et al., 20 NUCLEIC ACIDS RES. 5927-35 (1992)). Examples of variant FRT sequences are given by Jayaram (1985) and Senecoff et al. (1988), and an assay for Flp-mediated recombination on different substrates is described by Snaith et al. 180 GENE 225-7 (1996).

Accordingly, the process according to the invention can further comprise a step consisting in suppressing the first selection marker from the genome of said primary cell. In order to suppress said first selection marker, the cell is transfected by the gene coding the recombinase specific for the sequences allowing the suppression of the first selection marker. Methods and vectors able to transfer said gene into the cell are well known to the one skilled in the art, for example, the method disclosed in Example 4 of the present application can be used. Vectors previously described can also be used.

According to a preferred embodiment, the vector used in the process according to the invention comprises a second selection marker which is not surrounded by said homologous sequences, wherein said second selection marker is a negative selection marker. Said second selection marker is particularly useful when the vector, used in the process according to the invention, is circular. The presence of said second selection marker allows the destruction of the cells in which the homologous recombination process has led to the introduction of the section of the transfer vector that does not comprise the E1A nucleic acid sequence. When the vector is circular, the fact that the second selection marker is not surrounded by said homologous sequences means that the second selection marker and the E1A nucleic acid sequence are not positioned in the same section of the transfer vector, said section being delimited by the homologous sequences.

Accordingly, the process according to the invention can further comprise a step wherein the cells are cultivated in a medium which only allows the growth of the cells which have not incorporated the second selection marker. Said step can be made simultaneously with or separately from the step wherein said primary cells are cultivated in a medium which only allows the growth of the cells which have incorporated the first selection marker.

According to a preferred embodiment of the invention, the vector comprises a third selection marker wherein said third selection marker is a negative selection marker and wherein said third selection marker is located between the sequences allowing the suppression of the first selection marker. This means that the step consisting in suppressing the first selection marker will also lead to the suppression of the third selection marker. The presence of the third selection marker allows the destruction of the cells in which the first selection marker is present. When the vector is circular, the fact that the third selection marker is located between the sequences allowing the suppression of the first selection marker means that the third selection marker and the first selection marker are positioned in the same section of the transfer vector, said section being delimited by the sequences allowing the suppression of the first selection marker.

As used herein, the term negative selection marker notably refers to a gene encoding a product that kills the cells that carry the gene under certain conditions. These genes notably comprise “suicide gene”. The products encoded by these genes are able to transform a prodrug in a cytotoxic compound. Numerous suicide gene/prodrug pairs are currently available. There may be mentioned more particularly the pairs:
herpes simplex virus type I thymidine kinase (HSV-1 TK) and acyclovir or ganciclovir (GCV) (Caruso et al., 90 PROC. NATL. ACAD. SCI. USA 7024-7028 (1993); Culver et al., 256 SCIENCE 1550-1552 (1992); Ram et al., 3 NAT. MED. 1354-1361 (1997));
cytochrome p450 and cyclophosphophamide (Wei et al., 5 HUMAN GENE THERAPY 969-978 (1994));
purine nucleoside phosphorylase from Escherichia coli (E. coli) and 6-methylpurine deoxyribonucleoside (Sorscher et al., 1 GENE THERAPY 233-238 (1994));
guanine phosphoribosyl transferase from E. coli and 6-thioxanthine (Mzoz and Moolten, 4 HUMAN GENE THERAPY 589-595 (1993));
cytosine deaminase (CDase) and 5-fluorocytosine (5FC);
FCU1 and 5-fluoro-cytosine (5FC) (WO 99/54481); and
FCU1-8 and 5-fluoro-cytosine (5FC) (WO 2005/007857).

Said third selection marker allows the selection of the cells in which the suppression of the first selection marker has occurred. Accordingly, the process according to the invention can further comprise a step in which said cell is cultivated in a medium which does not allow the growth of the cell comprising the third selection marker. For example, a medium, which does not allow the growth of the cells comprising FCU1 as a third selection marker, comprises 5-Fluorocytosine.

The first, second, and third selection markers can be used separately. For example, the vector used in the process according to the invention can comprise the first and the third selection markers but not the second one, or the second and the third selection markers but not the first one.

According to a preferred embodiment of the invention, the E1A nucleic acid sequence, the first, the second, and/or the third selection marker are placed under the control of the elements necessary for their expression in the cell to be immortalized.

The elements necessary for the expression consist of the set of elements allowing the transcription of the nucleotide sequence to RNA and the translation of the mRNA to a polypeptide, in particular the promoter sequences and/or regulatory sequences which are effective in said cell, and optionally the sequences required to allow the excretion or the expression at the surface of the target cells for said polypeptide. These elements may be regulatable or constitutive. Of course, the promoter is adapted to the vector selected and to the host cell. There may be mentioned, by way of example, the eukaryotic promoters of the genes PGK (Phospho Glycerate Kinase), MT (metallothionein; Mclvor et al., 7 MOL. CELL BIOL. 838-848 (1987)), α-1 antitrypsin, CFTR, the promoters of the gene encoding muscle creatine kinase, actin pulmonary surfactant, immunoglobulin or β-actin (Tabin et al., 2 MOL. CELL BIOL. 416-436 (1982)), SRa (Takebe et al., 8 MOL. CELL. 466-472 (1988)), the SV40 virus (Simian Virus) early promoter, the RSV (Rous Sarcoma Virus) LTR, the MPSV promoter, the TK-HSV-1 promoter, the Cytomegalovirus (CMV) early promoter. The CMV early promoter is most particularly preferred.

The present invention more particularly relates, but is not limited to, a process for immortalizing a cell comprising:

transferring into an avian cell a vector comprising:
an E1A nucleic acid sequence surrounded by homologous sequences;
a first selection marker wherein said first selection marker is a positive selection marker and wherein said first selection marker is surrounded by said homologous sequences;
sequences allowing the suppression of the first selection marker;
a second selection marker which is not surrounded by said homologous sequences, wherein said selection marker is a negative selection marker; and
a third selection marker wherein said third selection marker is a negative selection marker and wherein said third selection marker is located between the sequences allowing the suppression of the first selection marker;
cultivating said cells in a medium which only allows the growth of the cells which have incorporated the first selection marker;
cultivating said cells in a medium which does not allow the growth of the cells which have incorporated the second selection marker;
excluding the first selection marker from the genome of said cell; and
cultivating said cell in a medium which does not allow the growth of the cells comprising the third selection marker.

According to a specific embodiment, the present invention also relates to an immortalized avian cell line comprising a E1A nucleic acid sequence as set forth in SEQ ID NO:1 and a nucleic acid sequence coding a recombinant telomerase reverse transcriptase as set forth in SEQ ID NO:3, selected in the group consisting of:
an immortalized avian cell line deposited at the European Collection of Cell Cultures (ECACC) under accession number 09070701,
an immortalized avian cell line deposited at the ECACC under accession number 09070702,
an immortalized avian cell line deposited at the ECACC under accession number 09070703, and
derivatives thereof.

Derivatives of the deposited Cairina moschata immortalized avian cell lines of the invention refer to Cairina moschata immortalized avian cell lines derived from the deposited ones by, for example:
subcloning of the said deposited Cairina moschata immortalized avian cell line (ECACC 09070701, ECACC 09070702 or ECACC 09070703);
adaptation of the said deposited Cairina moschata immortalized avian cell line (ECACC 09070701, ECACC 09070702 or ECACC 09070703) to a specific culture medium (e.g., medium allowing the growth of the cell line in suspension) and/or to specific culture conditions (e.g., temperature; % CO₂);
deletion or mutation of one or more genes of Cairina moschata involved in saccharide fucose modification (HARUE IMAI-NISHIYA et al., BMC BIOTECHNOLOGY (2007)) in order to produced non-fucosylated proteins, and more particularly non-fucosylated antibodies. According to a preferred embodiment, said derivatives of the deposited Cairina moschata immortalized avian cell line are obtained by deletion or mutation of α1,6-fucosyltransferase (FUT8) and/or GDP-mannose 4,6-dehydratase (GMD) genes;
deletion or mutation of one or more genes of Cairina moschata involved in interferon resistance in order to reduce the immune response of the cell line. According to a preferred embodiment, said derivatives of the deposited Cairina moschata immortalized avian cell line are obtained by deletion or mutation of gene(s) selected in the group consisting of STAT1 gene, STAT2 gene, STAT3 gene and STAT5 gene;
overexpression of one or more Cairina moschata's anti-apoptotic gene(s), or transformation with one or more exogenous anti-apoptotic gene(s) in order to render the cell line more resistant to the culture conditions, in particular for maintaining confluence. According to a preferred embodiment, said anti-apoptotic gene(s) is/are selected in the group consisting of p19E1B human adenovirus gene, bcl-2 gene, mcl-1 gene, Bcl-xL gene, Bcl-w gene, al gene, ICP34.5 herpes simplex virus gene, and p35 baculovirus gene;
overexpression of one or more Cairina moschata's genes involved in controlling the cell cycle using vectors which are suitable for increasing the rate of proliferation. According to a preferred embodiment, said gene(s) involved in controlling the cell cycle, is/are selected in the group consisting of p53 gene, p21 gene, p27 gene, and p57 gene;
modifying the viral sensitivity spectrum of the cell line by transformation with one or more genes which encode receptors for the viruses of interest, with a view to multiplying these viruses. According to a preferred embodiment, said gene(s) which encode receptors for the viruses of interest, is/are gene(s) which encode measles virus CD46 receptor.

The cell according to the invention can further comprise one or more nucleic acid sequence(s) allowing the propagation of a defective virus. “Defective virus” refers to a virus in which one or more viral gene necessary for its replication are deleted or rendered nonfunctional. The term “nucleic acid sequence allowing the propagation of a defective virus” refers to a nucleic acid sequence supplying in trans the function(s) which allows the replication of the defective virus. In other words, said nucleic acid sequence(s) code(s) the proteins(s) necessary for the replication and encapsidation of said defective virus.

The cell according to the invention can also comprise a nucleic acid sequence coding a substance of interest. As used herein, a substance of interest may include, but is not limited to, a pharmaceutically active protein, for example growth factors, growth regulators, antibodies, antigens, their derivatives useful for immunization or vaccination and the like, interleukins, insulin, G-CSF, GM-CSF, hPG-CSF, M-CSF or combinations thereof, interferons, for example, interferon-α, interferon-β, interferon-γ, blood clotting factors, for example, Factor VIII, Factor IX, or tPA or combinations thereof. “Substance of interest” also refers to industrial enzymes, for example for use within pulp and paper, textile modification, or ethanol production. Finally, “substance of interest” also refers to protein supplement or a value-added product for animal feed.

According to a preferred embodiment of the invention, the cell according to the invention also comprises a nucleic acid sequence coding a recombinant telomerase reverse transcriptase and more preferably, the recombinant telomerase reverse transcriptase described in EP-06360047.2. In a preferred embodiment of the invention described in EP-06360047.2, nucleic acid sequence coding the recombinant telomerase reverse transcriptase has at least 70%, more preferably at least 90%, and more preferably at least 95% nucleic acid sequence identity to the nucleic acid sequence set forth in SEQ ID NO:2 of EP-06360047.2. Preferred nucleic acid sequence coding the recombinant telomerase reverse transcriptase described in EP-06360047.2 is as set forth in SEQ ID NO:2 of EP-06360047.2. Telomerase reverse transcriptase nucleic acid sequence SEQ ID NO:2 described in EP-06360047.2 corresponds to telomerase reverse transcriptase nucleic acid sequence SEQ ID NO:3 in the present invention. As a consequence, according to a preferred embodiment of the invention, the cell according to the invention also comprises a nucleic acid sequence coding a recombinant telomerase reverse transcriptase and more preferably a nucleic acid sequence coding a recombinant telomerase reverse transcriptase having at least 70%, more preferably at least 90%, and more preferably at least 95% nucleic acid sequence identity to SEQ ID NO:3. More preferably, nucleic acid sequence coding a recombinant telomerase reverse transcriptase is as set forth in SEQ ID NO:3 (dTERT).

The cells obtained by the process according to the invention, the cell of the invention and the cells derived thereof are notably useful for the replication of a virus. Said viruses can be live, attenuated, recombinant, or not. More preferably, said cells are particularly useful for the replication of poxvirus (vaccinia virus, in particular MVA, canarypoxvirus, etc.), an adenovirus, a retrovirus, an herpesvirus, an alphavirus, a foamy virus, an adenovirus-associated virus, a flavivirus or an influenza virus.

Retroviruses have the property of infecting, and in most cases integrating into, dividing cells and in this regard are particularly appropriate for use in relation to cancer. A recombinant retrovirus generally contains the LTR sequences, an encapsidation region and the nucleotide sequence according to the invention, which is placed under the control of the retroviral LTR or of an internal promoter such as those described below. A retroviral vector may contain modifications, in particular in the LTRs (replacement of the promoter region with a eukaryotic promoter) or the encapsidation region (replacement with a heterologous encapsidation region, for example the VL30 type) (see FR-9408300 and FR-9705203).

An adenoviral vector can lack all or part of at least one region which is essential for replication and which is selected from the E1, E2, E4, and L1 L5 regions. A deletion of the E1 region is preferred. However, it can be combined with (an)other modification(s)/deletion(s) affecting, in particular, all or part of the E2, E4, and/or L1 L5 regions. By way of illustration, deletion of the major part of the E1 region and of the E4 transcription unit is very particularly advantageous. For the purpose of increasing the cloning capacities, the adenoviral vector can additionally lack all or part of the non-essential E3 region. According to another alternative, it is possible to make use of a minimal adenoviral vector which retains the sequences which are essential for encapsidation, namely the 5′ and 3′ ITRs (Inverted Terminal Repeat), and the encapsidation region. The various adenoviral vectors, and the techniques for preparing them, are known (see, e.g., Graham and Prevect, 7 METHODS IN MOLECULAR BIOLOGY 109-128; Ed: E. J. Murey, The Human Press Inc (1991)).

Poxvirus family comprises viruses of the Chordopoxvirus and Entomopoxvirus subfamilies. Among these, the poxvirus according to the invention is preferably chosen from the group comprising Orthopoxviruses, Parapoxviruses, Avipoxviruses, Capripoxviruses, Leporipoxviruses, Suipoxviruses, Molluscipoxviruses, and Yatapoxviruses. According to a more preferred embodiment, the poxvirus of the invention is an orthopoxvirus. Sequences of the genome of various Poxyiridae are available in the art, for example, the VV Western reserve, VV Copenhagen (VV-COP; GOEBEL et al. (1990); Modified Vaccinia virus Ankara, Cowpoxvirus, Canarypoxvirus, Ectromelia virus, Myxoma virus genomes are available in Genbank (accession number NC_{—}006998, M35027, U94848, NC_{—}003663, NC_{—}005309, NC_{—}004105, NC_{—}001132 respectively). The Orthopoxvirus is preferably a vaccinia virus and more preferably a modified vaccinia virus Ankara (MVA), in particular MVA 575 (ECACC V00120707) and MVA-BN (ECACC V00083008).

The term “recombinant virus” refers to a virus comprising an exogenous sequence inserted in its genome. As used herein, an exogenous sequence refers to a nucleic acid which is not naturally present in the parent virus.

In one embodiment, the exogenous sequence encodes a molecule having a directly or indirectly cytotoxic function. By “directly or indirectly” cytotoxic is meant that the molecule encoded by the exogenous sequence may itself be toxic (for example, ricin, tumor necrosis factor, interleukin-2, interferon-gamma, ribonuclease, deoxyribonuclease, Pseudomonas exotoxin A) or it may be metabolised to form a toxic product, or it may act on something else to form a toxic product. The sequence of ricin cDNA is disclosed in Lamb et al. (148 EUR. J. BIOCHEM. 265-270 (1985)) incorporated herein by reference.

In a preferred embodiment of the invention, the exogenous sequence is a suicide gene. A suicide gene encodes a protein able to convert a relatively non-toxic prodrug to a toxic drug. For example, the enzyme cytosine deaminase converts 5-fluorocytosine (5FC) to 5-fluorouracil (5FU) (Mullen et al. 89 PNAS 33 (1992)); the herpes simplex enzyme thymidine kinase sensitises cells to treatment with the antiviral agent ganciclovir (GCV) or aciclovir (Moolten, 46 CANCER RES. 5276 (1986); Ezzedine et al., 3 NEW BIOL 608 (1991)). The cytosine deaminase of any organism, for example E. coli or Saccharomyces cerevisiae, may be used.

Thus, in a more preferred embodiment of the invention, the gene encodes a protein having a cytosine deaminase activity and even more preferably a protein as described in WO 2005/007857 and WO 99/54481.

In a further embodiment the exogenous gene encodes a ribozyme capable of cleaving targeted RNA or DNA. The targeted RNA or DNA to be cleaved may be RNA or DNA which is essential to the function of the cell and cleavage thereof results in cell death or the RNA or DNA to be cleaved may be RNA or DNA which encodes an undesirable protein, for example an oncogene product, and cleavage of this RNA or DNA may prevent the cell from becoming cancerous.

In a still further embodiment the exogenous gene encodes an antisense RNA.

By “antisense RNA” we mean an RNA molecule which hybridises to, and interferes with the expression from a mRNA molecule encoding a protein or to another RNA molecule within the cell such as pre-mRNA or tRNA or rRNA, or hybridises to, and interferes with the expression from a gene.

In another embodiment of the invention, the exogenous sequence replaces the function of a defective gene in a target cell. There are several thousand inherited genetic diseases of mammals, including humans, which are caused by defective genes. Examples of such genetic diseases include cystic fibrosis, where there is known to be a mutation in the CFTR gene; Duchenne muscular dystrophy, where there is known to be a mutation in the dystrophin gene; sickle cell disease, where there is known to be a mutation in the HbA gene. Many types of cancer are caused by defective genes, especially protooncogenes, and tumor-suppressor genes that have undergone mutation.

Examples of protooncogenes are ras, src, bcl and so on; examples of tumor-suppressor genes are p53 and Rb.

In a further embodiment of the invention, the exogenous sequence encodes a Tumor Associated Antigen (TAA). TAA refers to a molecule that is detected at a higher frequency or density in tumor cells than in non-tumor cells of the same tissue type. Examples of TAA include, but are not limited to, CEA, MART-1, MAGE-1, MAGE-3, GP-100, MUC-1, MUC-2, pointed mutated ras oncogene, normal or point mutated p53, overexpressed p53, CA-125, PSA, C-erb/B2, BRCA I, BRCA II, PSMA, tyrosinase, TRP-1, TRP-2, NY-ESO-1, TAG72, KSA, HER-2/neu, bcr-abl, pax3-fkhr, ews-fli-1, survivin, and LRP. According to a more preferred embodiment the TAA is MUC1.

The recombinant virus can comprise more than one exogenous sequence and each exogenous sequence can encodes more than one molecule. For example, it can be useful to associate in a same recombinant poxvirus, an exogenous sequenced coding a TAA with an exogenous sequence coding a cytokine.

In another embodiment of the invention, the exogenous gene encodes an antigen. As used herein, “antigen” refers to a ligand that can be bound by an antibody; an antigen need not itself be immunogenic.

Preferably the antigen is derived from a virus such as for example HIV-1, (such as gp120 or gp 160), any of Feline Immunodeficiency virus, human or animal herpes viruses, such as gD or derivatives thereof or Immediate Early protein such as ICP27 from HSV1 or HSV2, cytomegalovirus (such as gB or derivatives thereof), Varicella Zoster Virus (such as gpl, II or III), or from a hepatitis virus such as hepatitis B virus for example Hepatitis B Surface antigen or a derivative thereof, hepatitis A virus, hepatitis C virus (preferentially non structural protein from genotype 1 b strain such as strain JA) and hepatitis E virus, or from other viral pathogens, such as Respiratory Syncytial Virus, Human Papilloma Virus (preferentially the E6 and E7 protein from the HPV16 strain) or Influenza virus, or derived from bacterial pathogens such as Salmonella, Neisseria, Borrelia (for example OspA or OspB or derivatives thereof), or Chlamydia, or Bordetella for example P.69, PT and FHA, or derived from parasites such as plasmodium or Toxoplasma.

The invention further relates to the use of any of said deposited Cairina moschata immortalized avian cell lines (ECACC 09070701, ECACC 09070702 and ECACC 09070703) and any derivative thereof, for producing biologics including viruses and proteins and related methods.

According to one embodiment, the present invention concerns a method for producing a virus, comprising the steps of infecting said immortalized avian cell line with a virus to be produced; cultivating the said infected cell line under conditions which are enabling virus amplification; and recovering the produced viruses; wherein said immortalized avian cell line is selected in the group consisting of immortalized cell line deposited at the European Collection of Cell Cultures (ECACC) under accession number 09070701, an immortalized cell line deposited at the ECACC under accession number 09070702 and an immortalized cell line deposited at the ECACC under accession number 09070703 and derivatives thereof. The method can also comprise additional step(s) such as the purification of the recovered viruses.

In a preferred embodiment, the virus to be produced is a Poxiviridae or an Influenza virus. Said Poxyiridae is preferably an Orthopoxvirus selected from the group consisting of Vaccinia virus strain Copenhagen (VV-COP) and Modified Vaccinia virus Ankara (MVA). Said Influenza virusis preferably of type A or of type B.

In a preferred method of the invention, the immortalized cell line is infected at a temperature comprised between 30° C. and 37° C., and more preferably at 37° C. Infection is performed in an appropriate cell culture medium which can be the same or different from the culture medium used subsequently during the amplification step. Suitable culture media include Dulbecco's Modified Eagle's Medium (DMEM, Invitrogen) or Basal Medium Eagle (BME, Invitrogen) which can be optionally supplemented with e.g. serum (e.g. Fetal Calf Serum (FCS)) and/or amino acid(s) (e.g. L-Glutamine). The cell culture medium can also be a medium free from animal product. Many media free from animal product have been already described and some of them are commercially available such as for instance 293 SFM II; 293-F Cells, SFM Adapted; 293-H Cells, SFM Adapted; 293fectin™ Transfection Reagent; CD 293 ACT™; CD 293 Medium; FreeStyle™ 293 Expression System; FreeStyle™ 293 Medium; FreeStyle™ 293-F Cells, SFM Adapted; VP-SFM; VP-SFM ACT™; Adenovirus Expression Medium (AEM) Growth Medium for PER. C6® Cells; CD 293 ACT™; CD 293 Medium; SFM Adapted; EPISERF® Medium; OptiPro™ SFM (all available from Invitrogen).

The infected cell line is then cultivated under conditions which are enabling virus amplification meaning that the viral genome is transcribed, translated into viral proteins and packaged into infectious viral particles. Preferably, the infected cell line is cultured between 1 and 7 days once infected. The infected cell line can be cultivated as adherent cells to surfaces or in suspension, in presence or absence of carriers. Cell culture can be done for instance in dishes, roller bottles or in bioreactors, using batch, fed-batch, continuous systems, hollow fiber, and the like.

The viruses produced are then recovered from the supernatant and/or from the cells. When the produced viruses are recovered from the cells (i.e. from the cells only, or from the cells and from the supernatant), the step of recovering of the viruses produced can comprise a step allowing the disruption of the cell membrane. This step leads to the liberation of the viruses from the cells. The disruption of the cell membrane can be induced by various techniques well known by the one skilled in the art. These techniques comprise but are not limited to freeze/thaw, hypotonic lysis, detergent-mediated lysis, sonication (by using a sonicator) and microfluidization (by using a microfluidizer). Sonicators are commercially available from e.g. Heraeus PSP, Biologics, Misonix or GlenMills. Preferred sonicators used according to the present invention are SONITUBE 20 kHz type SM 20-120-3 and SONITUBE 35 kHz type SM 35-400-3 (Heraeus PSP). Microfluidizers are commercially available from e.g. Microfluidics Corporation. The avian cell membrane can also be disrupted by using a using a SLM Aminco French press. The cell membrane can also be disrupted by using a high speed homogenizer. High speed homogenizers are commercially available from e.g. Silverson Machines or Ika-Labotechnik.

According to specific embodiment of the invention, the recovered viruses can be purified. Purification of the viruses produced can comprise for instance one or more of the following steps whatever the order of such steps such as clarification step allowing under suitable conditions the withdrawal of the cellular debris (Said clarification can be performed by e.g. depth filtration), a concentration step which can be performed by e.g. microfiltration or ultrafiltration; and/or a chromatography step, e.g. using a ion exchange adsorbent, gel filtration, hydroxyapatite, etc. with a specific preference for anion exchange, The functional groups of the anion exchange adsorbent can be primary, secondary, tertiary or quaternary amino group such as for instance dimethylaminoethyl (DMAE), diethylaminoethyl (DEAE), trimethylaminoethyl (TMAE), triethylaminoethyl (TEAE), the group —R—CH(OH)—CH₂—N+—(CH₃)₃ (also named Q group; see Streamline® resins, Pharmacia).

The present invention also relates to a method for producing an orthopoxvirus comprising the steps of (a) infecting an immortalized avian cell line of the invention with an Orthopoxvirus; (b) cultivating the infected avian cell line under conditions which are enabling virus amplification; (c) recovering said Orthopoxvirus from the culture supernatant and/or the packaging cells; and (d) optionally purifying said recovered Orthopoxvirus.

As described in Examples 4 and 5, preferred cell culture medium used for the production of Orthopoxvirus using the avian cell lines of the invention is BME (Invitrogen) supplemented with FCS and L-Glutamine. Infection is preferably performed at a low MOI which is preferably comprised between about 0.0001 and 0.1. More particularly, when the produced Poxyiridae is VV-COP, the MOI is more preferably about 0.0001 as described in Example 5. When the produced Poxyiridae is a MVA, the MOI is more preferably about 0.05 as described in Example 4. Preferably, before recovering the Orthopoxviruses, the infected cells are incubated in presence of one or more nucleases. Monovalent salts are added to the recovered Orthopoxviruses under suitable conditions to inhibit the nuclease(s) activity. The produced Orthopoxviruses can then be purified according to the any method known in the art, for example by chromatographic method, and especially anion exchange chromatography in association with clarification, diafiltration, etc.

The present invention also relates to a method for producing an influenza virus comprising the steps of (a) infecting an immortalized avian cell line with an influenza virus; (b) cultivating the infected avian cell line under conditions which are enabling virus amplification; (c) recovering said influenza virus from the culture supernatant and/or the packaging cells; (d) optionally purifying said recovered influenza virus.

Said Influenza virus (also called Flu viruses) can be of any type (e.g. A, B or C) and any subtype. “Subtypes of influenza type A virus” include the different combinations of HA and NA proteins possible. 19 classes of NA proteins (classified H1-H15) and 9 classes of NA proteins (classified N1-N9) have been identified in influenza type A viruses. As examples, subtypes of influenza type A virus can be H1N1 virus, H1N2 virus, H3N2 virus, H3N8 virus, H5N1 virus, H7N2 virus, H7N3 virus, H7N7 virus and H9N2 virus. Further information on the nucleotide sequences of the genomes of flu viruses can be obtained from publicly accessible gene databases such as www.flugenome.org/. Further information on the nucleotide sequences of the genomes of influenza type A viruses can also be obtained from publicly accessible gene databases such as GenBank, EMBL or LANL: e.g. J02144; J02146; J02148; J02151; V00603; V01099; V01104; V01106. Further information on the nucleotide sequences of the genomes of influenza type B viruses can also be obtained from publicly accessible gene databases such as GenBank, EMBL or LANL: e.g. J02094; J02095; J02096; K00423; K01395; M20168; M20170; M20172. Further information on the nucleotide sequences of the genomes of influenza type C viruses can also be obtained from publicly accessible gene databases such as GenBank, EMBL or LANL: e.g. K01689; M10087; M17700. Further information regarding attenuated flu virus vaccines can be found in HICKLING J. (A review of production for influenza virus vaccines, and their suitability for deployment in developing countries for influenza pandemic preparedness, Initiative for vaccine research, World Health Organisation (WHO) (2006); www.who.int/vaccine) and RUDENKO L. G. et al. (Vaccine 19(2-3), 308-318 (2000)). Further information regarding attenuated cold-adapted and temperature-sensitive flu virus vaccine such as for instance FluMist® (Medlmmune Vaccines) can be found in GLEZEN W. (Expert Rev. Vaccines 3(2):131-9 (2004)).

Example 6 describes a preferred method for producing influenza viruses from the cell line deposited at the European Collection of Cell Cultures (ECACC) under accession number 070970702.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Establishment of an Immortalized Avian Cell Line Comprising an EIA Nucleic Acid Sequence

A. Plasmid Constructs

A-1. Plasmid Constructs for Random Insertion.

A plasmid sharing no specific sequence of homology with the Cairina moschata genome (plasmid E1A) has been used for this purpose (FIG. 1).

A-2. Plasmid Constructs for Targeted Insertion.

A plasmid comprising two 5 kb fragments homologous to the Cairina moschata HPRT gene surrounding the E1A nucleic acid sequence (SEQ ID NO:1) and two selection markers has been constructed. The HPRT gene encoding for the hypoxanthine guanine phosphoryl transferase has been selected as an adequate site for the constitutive expression of the E1A nucleic acid sequence.

These two selection marker are the FCU1 gene (Erbs et al. Cancer Res. 2000. 15. 60.:3813-22) under the control of a CMV promoter (Thomsen et al. PROC. NATL. ACAD. SCI. USA. 81. 3:659-631984) and the Neomycin (or Puromycin) resistance gene placed under the control of a SV40 promoter. Neomycin (or Puromycin) resistance and FCU-1 expression cassette are surrounded by Sce1 cleavage sites that allow the elimination of the selection cassettes from the final cell line. Outside of the HPRT gene arms is inserted a selection marker coding the HSVTK driven by an RSV promoter (FIG. 2).

B. Preparation of CEC Batch from Cairina moschata Eggs and Subpopulations Description

B.1 Preparation of CEC Batch from 12 Old Cairina moschata Eggs and Subpopulations Description.

25 fertilized SPF eggs are incubated at 37.5° C. Eggs are opened after 12 days incubation following available protocol.

23 embryos are minced, washed once in Phosphate Buffered Saline-Dulbecco (PBS) and dissociated in TrypLE Select (Invitrogen) 5 hours at room temperature.

After low speed centrifugation cells are resuspended in Basal Medium Eagle (MBE) supplemented with 10% fetal calf serum (FCS), gentamycine 0.04 g/L, seeded in 500 cm² triple flasks and incubated at 37° C. 5% CO₂.

After 24 h the confluent cells are removed from the flasks using TrypLE Select (5 mL/triple flask), part of the cells were reseeded in 175 cm² flasks for second passage. The remaining cells were concentrated at 10⁷ cell/mL in appropriate media (60% BME, 30% FCS and 10% DMSO) and frozen in a isopropyl alcool regulated container (NALGENE.®. “Mr. Frosty” 1° C. freezing. Container) at −80° C. prior to transfer in liquid azote for long term storage, constituting the initial cell bank (50×1,5.10⁷ cells/vial, 44×1.10⁷ cells/vial).

Cells remained in culture are passaged classically up to 18 passages, during the 3 first passages non attached cells are collected by low centrifuging the conditioned media, reseeded and further passaged in the same way as the initial culture.

Subpopulations, displaying characteristic different morphological features, have been reproducibly isolated during the culture's lifespan.

B.2. Preparation of CEC Batch from 19 old Cairina moschata Eggs and Subpopulations Description.

29 fertilized SPF Cairina Moschata eggs obtained from AFFSSA Ploufragan are incubated at 37.5° C. in humid atmosphere.

Eggs are opened after 19 days and embryos sterilely extracted. 20 embryos are beheaded, limbs removed as well as the liver used for other cell preparation. The embryonic torsi are minced, washed once in PBS Dulbecco (Sigma, Ref. D8537, Lot 46K2428) and dissociated in 500 mL TrypLE Select (Gibco, Ref. 12563, Lots 1319986 and 1339844) 2 hours at 37° C.

After 5 minutes 2000 rpm centrifugation cells are resuspended in BME (Basal Medium Eagle, Gibco, Ref. 41010, Lot 8270) supplemented with 10% fetal calf serum (JRH, Ref. 12003-1000M, Lot. 5A0102, Code TG P4001Q), gentamycin 0.04 g/L and L-Glutamine 4 mM. A final volume of 1.5 L (1.9.10⁶ cell/mL) suspension is seeded in 10 triple flasks (500 cm²) and incubated at 37° C. 5% CO₂.

After 24 h the confluent cells are washed with PBS and removed from the flasks using TrypLE Select. Cells are counted and centrifuged 4-5 minutes at 2,000 rpm. The pellet is concentrated at 5.10⁶ or 10⁷ cell/mL in appropriate media (60% BME, 30% FCS and 10% DMSO). The suspension is filled in cryovials (Nunc) and frozen at −80° C. with a meanwhile 2 h step at −20° C., prior to transfer in liquid nitrogen for long term storage, constituting the primary cell bank (110 cryovials, 10⁷ cells/vial) of CETC19p1 (Duck Torso Embryonic Cells, 19 days old embryos, passage 1).

In a preferred embodiment of the present invention, the preparation of CEC batch from Cairina moschata eggs is performed according to alternative B.2. (from 19 old Cairina moschata eggs).

C. Methods of Transfection

A large number of transfection methods are known in the art to introduce a vector capable of directing expression of a nucleotide sequence of interest. A non limiting list of these methods is listed hereafter: CaPO₄ precipitation, electroporation, lipofectin transfection method. A given example is based on CaPO₄ precipitation procedure. Cells should be around 80-50% confluency. The medium is change two hours before CaPO₄/DNA addition. The 30 μg DNA is resuspended in 31 μl 2M CaCl₂-161.3 mM Tris pH 7.6. H₂O is added to a final volume of 0.5 ml.

Then, 2 alternatives:

a) Per transfection, 0.5 ml of 2×HEBS is distributed in 15 ml sterile Falcon tube and the DNA solution is added drop wise while gently vortexing or bubbling the DNA solution in. The solution should become milky. The mix is let stand at room temperature for 10-30 min. Then pipette in and out once with sterile pipette in tissue culture cabinet to break up flakes and apply drop wise to cells. Cells are then incubated between 6 hours to overnight at 37° C. A fine precipitate should cover the cell surface. In order to complete the transfection procedure warm up to 37° C. the glycerol shock solution. The medium is aspirate off, 5 ml BME is added to wash the cell layer, the medium is then aspirate off and 1 ml glycerol shock solution is added for 2 min or less. Subsequently 10 ml BME are added gently to dilute the glycerol and BME-glycerol is completely removed. 10 ml of desired medium is then added and plates are incubated at the appropriate temperature. or

b) Per transfection, 0.5 ml of 2×HEBS is distributed in 15 ml sterile Falcon tube and the DNA solution is added drop wise while gently vortexing or bubbling the DNA solution in. The solution should become milky. The mix is let stand at room temperature for 10-30 min. Then pipette in and out once with sterile pipette in tissue culture cabinet to break up flakes and apply drop wise to cells. A fine precipitate should cover the cell surface. Cells are then incubated between 6 hours to overnight at 37° C.

In a preferred embodiment of the present invention, the transfection (CaPO₄ precipitation) is performed according to alternative b).

D. Methods of Selection

D-1. Method of Selection for Random Insertion:

Selection pressure is applied 48 to 72 hours after transfection: cells are dissociated with TrypLE select, low speed centrifuged and reseeded in BME with FCS 10% and G418 800 μg/mL, preferably 500 μg/mL (and optionally Ganciclovir 25 μg/mL, preferably 10 μg/mL).

Cells are serially passaged until individual growing clones can be isolated.

D-2. Method of Selection for Targeted Insertion:

Selection pressure is applied 48 to 72 hours after transfection: cells are dissociated with TrypLE select, low speed centrifuged and reseeded in BME with FCS 10%; Ganciclovir 25 μg/mL, preferably 10 μg/mL; and G418 800 μg/mL, preferably 500 μg/mL (or Puromycin 0.5 μg/mL).

Cells are serially passaged until individual growing clones can be isolated.

Cell clones are subsequently transfected with a meganuclease I-SceI expression plasmid following the method described below.

To select the elimination of the selection markers 5-Fluorocytosine (5-FC) is applied 48 hours after transfection: cells are dissociated with TrypLE select, low speed centrifuged and reseeded in media with 5-FC concentration ranging from 10⁻³ to 10⁻⁷ M and maintained G418 (or Puromycin)/Ganciclovir selection (BME with FCS 10%; 5-FC Ganciclovir 25 μg/mL, preferably 10 μg/mL; and G418 800 μg/mL, preferably 500 μg/mL (or Puromycin 0.5 μg/mL) (FIG. 3).

### Example 2

### Establishment of an Immortalized Avian Cell Line Comprising an EIA Nucleic Acid Sequence and a Recombinant Telomerase Reverse Transcriptase Nucleic Acid Sequence

A. Plasmid Constructs

A-1. Plasmid Constructs for Random Insertion.

A plasmid sharing no specific sequence of homology with the Cairina moschata genome has been used for this purpose.

A-2. Plasmid Constructs for Targeted Insertion.

A plasmid (plasmid dTERT-E1A) comprising two 5 kb fragments homologous to the Cairina moschata HPRT gene surrounding the Cairina moschata telomerase reverse transcriptase gene (SEQ ID NO:3), the E1A nucleic acid sequence (SEQ ID NO:1) and two selection markers has been constructed. The HPRT gene encoding for the hypoxanthine guanine phosphoryl transferase has been selected as an adequate site for the constitutive expression of the E1A nucleic acid sequence.

These two selection marker are the FCU1 gene (Erbs et al. Cancer Res. 2000. 15. 60.:3813-22) under the control of a CMV promoter (Thomsen et al. P.N.A.S. 1984. 81. 3:659-63) and the Puromycin resistance gene placed under the control of a SV40 promoter. Puromycin resistance and FCU-1 expression cassette are surrounded by Sce1 cleavage sites that allow the elimination of the selection cassettes from the final cell line. Outside of the HPRT gene arms is inserted a selection marker coding the HSVTK driven by an RSV promoter (FIG. 4).

B. Preparation of CEC Batch from 19 Old Cairina moschata Eggs and Subpopulations Description

29 fertilized SPF Cairina Moschata eggs obtained from AFFSSA Ploufragan are incubated at 37.5° C. in humid atmosphere.

Eggs are opened after 19 days and embryos sterilely extracted. 20 embryos are beheaded, limbs removed as well as the liver used for other cell preparation. The embryonic torsi are minced, washed once in PBS Dulbecco (Sigma, Ref. D8537, Lot 46K2428) and dissociated in 500 mL TrypLE Select (Gibco, Ref. 12563, Lots 1319986 and 1339844)₂ hours at 37° C.

After 5 minutes 2000 rpm centrifugation cells are resuspended in BME (Basal Medium Eagle, Gibco, Ref. 41010, Lot 8270) supplemented with 10% fetal calf serum (JRH, Ref. 12003-1000M, Lot. 5A0102, Code TG P4001Q), gentamycin 0.04 g/L and L-Glutamine 4 mM. A final volume of 1.5 L (1.9.10⁶ cell/mL) suspension is seeded in 10 triple flasks (500 cm²) and incubated at 37° C. 5% CO₂.

After 24 h the confluent cells are washed with PBS and removed from the flasks using TrypLE Select. Cells are counted and centrifuged 4-5 minutes at 2000 rpm. The pellet is concentrated at 5.10⁶ or 10⁷ cell/mL in appropriate media (60% BME, 30% FCS and 10% DMSO). The suspension is filled in cryovials (Nunc) and frozen at −80° C. with a meanwhile 2 h step at −20° C., prior to transfer in liquid nitrogen for long term storage, constituting the primary cell bank (110 cryovials, 10⁷ cells/vial) of CETC19 μl (Duck Torso Embryonic Cells, 19 days old embryos, passage 1).

C. Methods of Transfection

A large number of transfection methods are known in the art to introduce a vector capable of directing expression of a nucleotide sequence of interest. A non limiting list of these methods is listed hereafter: CaPO₄ precipitation, electroporation, lipofectin transfection method. A given example is based on electroporation.

Transfection is performed using Amaxa's Nucleofector device and the Basic Fibroblast kit (Amaxa, Cat N° VPI-1002). Cells are centrifuged 10 min at 700 rpm (100 g) and resuspended in Basic Nucleofector Solution (100 μL per 10⁶ cells); 100 μL suspension are mixed with 3 to 6 μg DNA and transferred to a cuvette placed in the Nucleofector (U-12 program). After electroporation the sample is transferred to a 6 cm culture dish, filled with 5 mL culture media, preequilibrated in the 37° C./5% CO₂ incubator. After incubation over night at 37° C. 5% CO₂ culture media is renewed and incubation pursued.

D. Methods of Selection

D-1. Method of Selection for Random Insertion:

Selection pressure is applied 48 to 72 hours after transfection: cells are dissociated with TrypLE select, low speed centrifuged and reseeded in BME with FCS 10%, Ganciclovir 25 μg/mL, preferably 10 μg/mL; and G418 800 μg/mL, preferably 500 μg/mL.

Cells are serially passaged until individual growing clones can be isolated.

D-2. Method of Selection for Targeted Insertion:

Selection pressure is applied 48 to 72 hours after transfection: cells are dissociated with TrypLE select, low speed centrifuged and reseeded in BME with FCS 10%; Ganciclovir 25 μg/mL, preferably 10 μg/mL; and Puromycin 0.5 μg/mL.

Cells are serially passaged until individual growing clones can be isolated.

Cell clones are subsequently transfected with a meganuclease I-SceI expression plasmid following the method described below.

To select the elimination of the selection markers 5-Fluorocytosine (5-FC) is applied 48 hours after transfection: cells are dissociated with TrypLE select, low speed centrifuged and reseeded in media with 5-FC concentration ranging from 10⁻³ to 10⁻⁷ M and maintained Puromycin/Ganciclovir selection (BME with FCS 10%; 5-FC Ganciclovir 25 μg/mL, preferably 10 μg/mL; and Puromycin 0.5 μg/mL).

### Example 3

### ECACC 09070701, ECACC 09070702, and ECACC 09070703 Immortalized Cairina moschata Cell Lines

The Cairina moschata cell lines ECACC 09070701 (T17-17703B), ECACC 09070702 (T17-17703B2), and ECACC 09070703 (T17-17703A) were obtained from primary embryonic Cairina moschata cells as described in Example 2 by target insertion (electroporation) with the plasmid dTERT-E1A.

The cells lines arose between 210 and 260 days post-transfection, after a senescence/crisis phase, as the subpopulations ECACC 09070701, ECACC 09070702, and ECACC 09070703 entered a new continuous exponential growth phase. Corresponding growth curves (FIG. 8) show the populations' evolution until 456 days post-transfection. The numbers of population doublings (PDL) calculated for this period (ECACC 09070703: 138 PDL; ECACC 09070701: 131 PDL; ECACC 09070702: 166 PDL) are far beyond the Hayflick limit. The Cairina moschata cell lines ECACC 09070701 (T17-17703B), ECACC 09070702 (T17-17703B2), and ECACC 09070703 (T17-17703A) are consequently referred as immortalized cell lines.

The three Cairina moschata cell lines are distinct cell lines which display different morphologies: The Cairina moschata ECACC 09070703 cells have an epithelial like morphology (FIG. 7), whereas the Cairina moschata ECACC 09070701 and ECACC 09070702 cells form syncitia like layers (respectively FIG. 5 and FIG. 6). The cells of the three cell lines are small cells, growing in a stable static monolayer.

The three Cairina moschata cell lines also display different generation times: Average population doubling time (PDT) is 30 hours for ECACC 09070702, 35 hours for ECACC 09070701 and 47 hours for ECACC 09070703 (FIG. 9).

The cells of the three cell lines were tested negative for mycoplasma and microbial contaminations.

The population doubling level (PDL) refers to the number of cell generations

(biomass 2 fold increase). PDL calculation: PDL=Ln(final/initial cell number)/Ln(2);

The population doubling time (PDT), also called generation time, is the time needed for one population doubling. PDT calculation: PDT=Δt*Ln(2)/Ln(final/initial cell number).

### Example 4

### Production of Modified Vaccinia Virus Ankara (MVA)

MVA amplification capacities of Cairina moschata immortalized avian cell lines ECACC 09070701, ECACC 09070702, and ECACC 09070703 were evaluated and compared to primary chicken embryo fibroblasts (CEFs) usually used as substrate for MVA production. An MVA (Collection Nationale de Cultures de Microorganismes (CNCM) under depositary N₆O₂ 1-721) expressing eGFP was chosen in order to facilitate virus propagation follow up and titration. Cairina moschata immortalized avian cell lines ECACC 09070701, ECACC 09070702, ECACC 09070703 and CEFs were seeded at 2.10⁶ cells in T-flask of 25 cm² and cultivated for 24 hours in humid atmosphere at 37° C., 5% CO₂. The culture medium (Basal Medium Eagle (BME) supplemented with 10% Foetal Calf Serum (FCS) and 4 mM L-Glutamine) was then removed and cells infected at MOI 0.05 with 500 μL MVA virus diluted in PBS 1% cations, 1% FCS. After a 30 minutes adsorption step, remaining virus suspension was removed, cells washed once with PBS and 5 mL BME 10% FCS were then added to each flask. Fresh culture medium was added and cells replaced in the incubator. Virus was recovered by a freezing-thawing step from cells and supernatant after 0, 24, 48, 72 and 96 hours infection at 37° C., 5% CO₂. Recovered virus suspensions were sonicated prior to titration in order to avoid aggregates. Titrations were performed in triplicates on CEFs seeded in 6 cm culture dishes, infected with logarithmic virus dilutions and overlaid with agar. Plaque forming units of MVA, expressing eGFP, were visualized with fluorescent binocular and counted after 72 hours.

Results: As depicted in FIG. 10, the Cairina moschata immortalized avian cell lines ECACC 09070701, ECACC 09070702, and ECACC 09070703 were permissive to MVA and enabled MVA amplification comparable to the one obtained with classical CEF substrate. For all the tested avian cell lines, the maximal viral yield was reached 72 h post-infection and was ranging from 2,3.10⁷ to 5,7.10⁷ total pfu. Concordantly, clear cytopathic effect was observed in all cells (data not shown).

### Example 5

### Production of Vaccinia Virus Strain Copenhagen (VV-COP)

VV-COP amplification capacity of Cairina moschata immortalized avian cell lineECACC 09070702 was evaluated and compared to the reference primary chicken embryo fibroblasts (CEFs). A VV-COP expressing eGFP was chosen in order to facilitate virus propagation follow up and titration. Avian cell line ECACC 09070702 (at passage 50) and CEFs were seeded at 1.4×10⁷ cells in T-flask 175 and cultivated for 24 hours at 37° C., 5% CO₂ atmosphere. The culture medium (Basal Medium Eagle (BME) supplemented with 10% fetal calf serum (FCS), 4 mM L-Glutamine and 0.04 g/L gentamycin) was then removed and cells infected with the virus diluted in PBS supplemented with 1% FCS and cations (Merck mixture: magnesium acetate 100 μg/mL, calcium chloride 100 μg/mL) at low MOI 0.0001. After 30 min adsorption at room temperature, fresh culture medium was added and cells replaced in the incubator (at 37° C. and 5% CO₂). Cells were subsequently frozen after 24, 48, 72 and 96 h infection.

Virus was recovered following freezing-thawing step from cells and supernatant after 0, 24, 48, 72 and 96 hours infection. Recovered virus suspensions were sonicated prior to titration in order to dissociate virus aggregates before titration. Titration was performed in triplicates onto BHK21 cells in 6 well culture plates. Cells were seeded at 5×10⁵ cells per well in 2 mL DMEM (Gibco), 10% FCS and allowed to incubate during 24 h. Serial dilutions of VV solutions ranging from 10⁻² to 10⁻⁸ were made in PBS, 1% FCS and cations (Merck mixture. 250 μL of each dilution was added per well and contact between viral dilutions and cells was allowed for 30 min at room temperature. 2 mL of DMEM 10% FCS were subsequently added per well and cells incubated at 37° C. in 5% CO₂ atmosphere. 24H later, media was removed and the cells overlaid with 1 mL of a 1:1 vol/vol mixture of crystal violet (10%; Sigma) and red neutral (2 g/L; Merck). After a one hour staining at room temperature, plaque forming units (pfu) of VV-COP expressing eGFP, were visualized and counted with fluorescent binocular.

As depicted in FIG. 11, the Cairina moschata immortalized avian cell line ECACC 09070702 was permissive to VV-COP and enabled its amplification at a level comparable to the one obtained with CEF (2.7×10⁸ pfu and 6.8×10⁸ pfu, respectively). In both case, the maximal viral yield was reached 96 h post-infection.

This study showed that the Cairina moschata immortalized avian cell lines of the invention are good candidates for both MVA and VV-COP production.

### Example 6

### Production of Flu Virus

The capacity of Cairina moschata immortalized avian cell IineECACC 09070702 (T17-17703B2) was evaluated for the production of influenza viruses. Several influenza strain were tested influenza type A (A/Panama/2007/99 H3N2 strain) and influenza type B (B/Brisbane/60/2008). Infection and T17 cell permissivity were evaluated by quantification of viral intracellular neuraminidase.

Neuraminidase Assay (NA)

T17 cells were seeded into 96-well plates at 0.15×10⁵ cells per well and cultured in infection medium (BME supplemented with 2 mM L-glutamine, 100 U/mL penicillin, 100 μg/mL streptomycin and 0.5 μg/mL trypsin) until 100% confluence. Cells were infected with influenza viruses at a MOI of 1 or 0.1 by adding 25 μL per well of virus diluted in infection medium. Infection was allowed to proceed for 75 h (MOI 0,1) and 48 h (MOI 1) at 37° C., 5% CO2 after which 25 μL of supernatant were collected for the Neuraminidase activity test.

Standard fluorometric endpoint assays used to monitor NA activity was recently shown to be suitable to quantify influenza virus in a high-throughput screening test (Virol J. 2008 5: 109). Briefly, cell supernatants (25 μl) were transferred to a black 96-well plate and 75 μl of 2′-(4-methylumbelliferyl)-alpha-N-acetylneuraminic acid (MUNANA, Sigma Chemical Co.) to a final concentration of 50 μM were added. After incubation of the plate at 37° C. for 1 hr, 150 μl stop solution (0.05 M glycine, pH 10.4) was added to each well and the fluorescence read on a FluoStar Opima (BMG Labtech) with excitation and emission filters of 355 nm and 460 nm respectively. Relative fluorescence units (RFU) were corrected by subtracting specific blanks, ie medium with or without molecules.

Potential interference of test molecules on the NA enzymatic activity was tested by incubating the A/Moscow/10/99 (H3N2) viral stock diluted in DMEM (107.8 TCID50/mL final) with increasing concentrations of the test molecule (or DMEM for control) for 0.5 h at room temperature. Specific blanks were measured for each molecule. 25 μL were used for the NA test as described above and results were expressed as a ratio of corrected RFU of the sample to RFU of controls. Two independent experiments were performed in duplicate

Result

Measure of the neuraminidase activity showed that ECACC 09070702 cell line was permissive to influenza type A (A/Panama/2007/99 H3N2 strain; FIG. 12) and to influenza type B (B/Brisbane/60/2008; FIG. 13). Detection of the neuraminidase activity translates both that influenza replication is effective in ECACC 09070702 cell line and that viral neuraminidase is correctly processed and functional.

Each patent, patent application, publication, text, and literature article/report cited or indicated herein is hereby expressly incorporated by reference in its entirety for all purposes.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. An immortalized avian cell line as deposited at the European Collection of Cell Cultures (ECACC) under accession number 09070701 and derivatives thereof.

2. An immortalized avian cell line as deposited at the European Collection of Cell Cultures (ECACC) under accession number 09070702 and derivatives thereof.

3. An immortalized avian cell line as deposited at the European Collection of Cell Cultures (ECACC) under accession number 09070703 and derivatives thereof.

4. The immortalized avian cell according to claim 1, wherein it further comprises one or more nucleic acid sequence allowing the propagation of a defective virus.

5. The immortalized avian cell line according to claim 1, wherein it further comprises a nucleic acid sequence coding a substance of interest.

6. A method for replicating a virus, wherein said method comprises employing an immortalized avian cell line according to any one of claims 1 to 3.

7. The method according to claim 6, wherein said virus is a live virus, an attenuated virus, or a recombinant virus.

8. The method according to claim 6, wherein said virus is chosen from the group consisting of poxvirus, adenovirus, retrovirus, herpervirus, alphavirus, foamy virus, adenovirus-associated virus, flavivirus) and influenza virus.

9. The method according to claim 8, wherein said virus is a poxvirus.

10. The method according to claim 9, wherein said virus is a vaccinia virus.

11. The method according to claim 10, wherein said virus is a modified vaccinia virus Ankara (MVA).

12. The method according to claim 10, wherein said virus is a vaccinia virus strain Copenhagen.

13. The method according to claim 8, wherein said influenza virus is of type A

14. The method according to claim 13, wherein said influenza virus of type A is selected from the group consisting of H1N1 and H3N2 strains

15. The method accordinci to claim 8, wherein said influenza virus is of type B

16. The method according to claim 15, wherein said influenza virus of type B is selected from the group consisting of Brisbane and Florida strains.

17. The immortalized avian cell according to claim 2, wherein it further comprises one or more nucleic acid sequence allowing the propagation of a defective virus.

18. The immortalized avian cell according to claim 3, wherein it further comprises one or more nucleic acid sequence allowing the propagation of a defective virus.

19. The immortalized avian cell line according to claim 2, wherein it further comprises a nucleic acid sequence coding a substance of interest.

20. The immortalized avian cell line according to claim 3, wherein it further comprises a nucleic acid sequence coding a substance of interest.
